SEQUENCE LISTING (23 sequences)

SEQ ID NO: 1 - DNA, 20 bp, Artificial Sequence
  Primer, reverse sequence (5′-->3′)
agagtttgatcmtggctcag

SEQ ID NO: 2 - DNA, 22 bp, Artificial Sequence
  Primer, forward sequence (3′-->5′)
tacggytaccttgttacgactt

SEQ ID NO: 3 - DNA, 21 bp, Artificial Sequence
  Primer, reverse sequence (5′-->3′)
caggcctaacacatgcaagtc

SEQ ID NO: 4 - DNA, 18 bp, Artificial Sequence
  Primer, forward sequence (3′-->5′)
gggcggwgtgtacaagrc

SEQ ID NO: 5 - DNA, 24 bp, Artificial Sequence
  Primer, reverse sequence (5′-->3′)
gcatatcaataagcggaggaaaag

SEQ ID NO: 6 - DNA, 19 bp, Artificial Sequence
  Primer, forward sequence (3′-->5′)
ggtccgtgtttcaagacgg

SEQ ID NO: 7 - DNA, 19 bp, Artificial Sequence
  Primer, reverse sequence (5′-->3′)
tccgtaggtgaacctgcgg

SEQ ID NO: 8 - DNA, 20 bp, Artificial Sequence
  Primer, forward sequence (3′-->5′)
tcctccgcttattgatatgc

SEQ ID NO: 9 - DNA, 683 bp, Artificial Sequence
  Primer PP3/NL-1, reverse sequence
gggcttagggcacacctagcgataaaatatactaaacgggttcctcctttaaacattagcaccacccgaagcagaattctaacatttattagcgaaagcgcccagcaatcttgcctaaaggcattcgctgcgttcctcagtccaatccagagtatgataataggctataaacacctcgaaaggccacattcctttaattttttcctccaaatcaaactgtcgttggcaggcatagactgcaagtgcacccaaaacgaatttctaggttgattacaatcaaaccaatctggctcccaaacgttcccttttaacaatttcacatactgtttaactctcttttcaaagttcttttcatctttccctcacggtacttgttcgctatcggtctctcgcaaatatttagctttaggtgagatttaccacccaatttaggctgcattcccaaacaacctgactctttgaaaacatatcacaatagacaactacttagaccaaagacgggattctcaccctctatgatgccctgttccaaaggacttttttgtctagtgtatctggaaaatgtttctacagtttacaatccggtttagctaagccaaacaggttccaactttgagctctttcctcttcactcgccgttactagggaaatcattgttattttcttttcctcatatatggaat

SEQ ID NO: 10 - DNA, 687 bp, Artificial Sequence
  Primer PP3/NL-4, forward sequence
tggggcatttccctagtacggcgagtgagaggaagagctaaaagttggaacctgtttggcttagctaaaccggattgtaaactgtagaaacattttccagatacactagacaaaaaagtcctttggaacagggcatcatagagggtgagaatcccgtctttggtctaagtagttgtctattgtgatatgttttcaaagagtcaggttgtttgggaatgcagcctaaattgggtggtaaatctcacctaaagctaaatatttgcgagagaccgatagcgaacaagtaccgtgagggaaagatgaaaagaactttgaaaagagagttaaacagtatgtgaaattgttaaaagggaaccgtttggagccagattggcttgattgtaatcaacctagaattcgttttgggtgcacttgcagtctatgcctgccaacgacagtttgatttggaggaaaaaattaataggaatgtggcctttcgaggtgttatagcctattatcatactctggattggactgaggaacgcagcgaatgcctttaggcaagattgctgggcgctttcgctaataaatgttagaatttctgcttcgggtggtgctaatgtttaaaggaggaacccgtttagtatattttttattcgcttaggttgttggcttaatgactctaaatgacccgtctaaaaccgggga

SEQ ID NO: 11 - DNA, 1000 bp, Artificial Sequence
  Primer PP4/ITS1, reverse sequence
gggctaaatatcatatcttggcttgtccattattatctatttactgtgaactgtattattatttgacatttgagggatgttccaatgttataaggatagacattggaaatgttaaccgagtcataatcaggtttaggcctggtatcctattattatttaccaaatgaattcagaattaatattgtaacatagacctaaaaaatctataaaacaacttttaacaacggatctcttggttctcgcatcgatgaagaacgtagcaaagtgcgataactagtgtgaattgcatattcagtgaatcatcgagtctttgaacgcaacttgcgctcattggtattccaatgagcacgcctgtttcagtatcaaaacaaaccctctattcaacttttgttgtataggattattgggggcctctcgatctgtatagatcttgaaatccctgaaatttactaaggcctgaacttgtttaaatgcctgaacttttttttaatataaaggaaagctcttgtaattgactttgatggggcctcccaaataaatcttttttaaatttgatctgaaatcaggcgggattacccgctgaacttaagcatatctaaacgagaaaaaaaaaaaaatcctttaaataatcattaatctgggctggtccttaataatctatttacggtgaaatggtataattatttgacatttgaggggatgttccaatgtttataagggatagactttggaaaatgttaaccgagtcaaaatcgggtttaagcctgggtatccaattaattattttacaaaggaaattcaaattaaaattgtttacaaagaactaaaaaaatctattaaaaaacttttaacaacggaaactttgggtttctgcaccctttaaaaaaagtaacaaaatggcaataactagtggggaaattgcaaaattccctggaaatcacccggttcttttgaaagccacctgggcctcaatggggtttttcccatgaagcaagccccg

SEQ ID NO: 12 - DNA, 850 bp, Artificial Sequence
  Primer PP4/ITS2, forward sequence
tgatctcgcttccgcctgatttcagatcaatttaaaaaagatttatttgggaggccccatcaaagtcaatttacaagagctttcctttatattaaaaaaaagttcaggcatttaaacaagttcaggccttagtaaatttcagggatttcaagatctatacagatcgagaggcccccaataatcctatacaacaaaagttgaatagagggtttgttttgatactgaaacaggcgtgctcattggaataccaatgagcgcaagttgcgttcaaagactcgatgattcactgaatatgcaattcacactagttatcgcactttgctacgttcttcatcgatgcgagaaccaagagatccgttgttaaaagttgttttatagattttttaggtctatgttacaatattaattctgaattcatttggtaaataataataggataccaggcctaaacctgattatgactcggttaacatttccaatgtctatccttataacattggaacatccctcaaatgtcaaataataatacagttcacagtaaatagataataatggactagccaagattattgattatttaatgatccttccgcatctccccaccggaaaaaagaaaaaatcctttaaaaaaaccaataaaccggggccgggcccttaaaaattttttttccggtgaaatggtaaaaaaaaatttgaaatttggaagggaaggtttccaatgtttataaaggaaaagaactttgggaaaatgtttaaccgagttcaaaaaccgggttttagggccggggtatcccattaaaaaattttaccaaaggaaaatccccaaaataaaaatttggtgaacaaaaga

SEQ ID NO: 13 - DNA, 673 bp, Artificial Sequence
  Primer PP3/NL-1, reverse sequence
  misc_feature (577)..(577): n is a, c, g, or t
  misc_feature (591)..(592): n is a, c, g, or t
catcctaggtgtgagaggtcgacccccgcctaaggcacactgggttcctcagtcccccaagatgtatcccgcagagagctataacacagccgaaactgctaccttctctctaccattatccatcccggaaaactgatgctggcctgcaaaccgatggctcggcaagcaagtctgacttccagcgattcccttccaacaatttcacgtactgttaactctctttccaaagtgcttttcatctttccctcacggtacttgttcgctatcggtctcccgccaatatttagctttagatggaatttaccacccaatttgagctgcattcccaaacaactcgaaacttcgaaaatgtatcacaaatggctgggcgtccgcaccatatacgggggtctcttcactatgccgctatattccaaaaaacttgtgtgcggtccaacgcggaaaacatttctagagattacaactgggagaccgaaagagccagattataaatttgagctcgtcccgctcccctcgccgctacgaaggggaatccatgggcgtttcaattccccccctgaaaaaaaagcaaaaaaantcgggaaacccctnngtatactctggagcagtctcatgtactacgacatacagaactctcttgcagtatgtctactccctcgtggggaaaaaggac

SEQ ID NO: 14 - DNA, 687 bp, Artificial Sequence
  Primer PP3/NL-4, reverse sequence
tggggcatttccctagtacggcgagtgagaggaagagctcaaagttggaacctgtttggcttagctaaaccggattgtaaactgtagaaacattttccagatacactagacaaaaaagtcctttggaacagggcatcatagagggtgagaatcccgtctttggtctaagtagttgtctattgtgatatgttttcaaagagtcaggttgtttgggaatgcagcctaaattgggtggtaaatctcacctaaagctaaatatttgcgagagaccgatagcgaacaagtaccgtgagggaaagatgaaaagaactttgaaaagagagttaaacagtatgtgaaattgttaaaagggaaccgtttggagccagattggcttgattgtaatcaacctagaattcgttttgggtgcacttgcagtctatgcctgccaacgacagtttgatttggaggaaaaaattaataggaatgtggcctttcgaggtgttatagcctattatcatactctggattggactgaggaacgcagcgaatgcctttaggcaagattgctgggcgctttcgctaataaatgttagaatttctgcttcgggtggtgctaatgtttaaaggaggaacccgtttagtatattttttattcgcttaggttgttggcttaatgactctaaatgacccgtctaaaaccgggga

SEQ ID NO: 15 - DNA, 1260 bp, Artificial Sequence
  Primer PP4/ITS1, reverse sequence
acggaattggaaaatagtaagctcacattctcatcaaaagtgtttctggggaagttctgaccctgtgcacttgtttgggatagtaactctcgcaagagagcgaactcctattcacttataaacacaaagtctatgaatgtattaaattttataacaaaataaaactttcaacaacggatctcttggctctcgcatcgatgaagaacgcagcgaaatgcgataagtaatgtgaattgcagaattcagtgaatcatcgaatctttgaacgcaccttgcgctccatggtattccgtggagcatgcctgtttgagtgtcatgaatacttcaaccctcctctttcttaatgattgaagaggtgtttggtttctgagcgctgctggcctttacggtctagctcgttcgtaatgcattagcatccgcaatcgaacttcggattgacttggcgtaatagactattcgctgaggaattctagtcttcggactagagccgggttgggttaaaggaagcttctaatcagaatgtctacattttaagattagatctcaaatcaggtaggactacccgctgaacttaagcatatctaaacccgaaaggaaaggaatcattaggggaatataggaacgtccacttaacttgggagtccgaactctcactttctaaccctgtgcacttgttttgggatagttaactcttgcaagagagcgaactcctattcacttataaacacaaagtctatgaaaggtattaaaatttttataacaaaattaaaattttcacaacggaaactttggggctctggcatcgaataaaaaaaagcaacgaaaatgcggattagttaaatggtggaaatggcagaaaattcattggaaatcaccgaaaccttttgaacgcaccttggcgctcccggggtaattccctgggaaacaaggcccgtttttgaagttgttcaggaaaaaaacattcaaccctccccctttttcttaaaaaggaattggaaaaaaaaggtttttttttgggttttttcctgaaagccccttcctgggccctttttaaggggttttaaaccttgtttttgtttaaaagggccaaattaaaacaacccccccaattccaaaaaatttttcggaaaaattggaaacctttggggcccctttaaatttaaaaaaaaccaaaattttcccccttgaaaaggggaaaaaatttttctttggggtttccctttccgcgggaaacattaaaaaaaaagccccccggggggt

SEQ ID NO: 16 - DNA, 617 bp, Artificial Sequence
  Primer PP4/ITS2, forward sequence
aatgggaatttaggaggtccacttaacttgtcagtccgaactctcctttcgctcctgtgatcttgtttaacccaacccggctctagtccgaagactagaattcctcagcgaatagtctattacgccaagtcaatccgaagttcgattgcggatgctaatgcattacgaacgagctagaccgtaaaggccagcagcgctcagaaaccaaacacctcttcaatcattaagaaagaggagggttgaagtattcatgacactcaaacaggcatgctccacggaataccatggagcgcaaggtgcgttcaaagattcgatgattcactgaattctgcaattcacattacttatcgcatttcgctgcgttcttcatcgatgcgagagccaagagatccgttgttgaaagttttattttgttataaaatttaatacattcatagactttgtgtttataagtgaataggagttcgctctcttgcgagagttactatcccaaacaagtgcacagggttagaaagtgagagttcggactccaagttaagttggacgtcctatattcactaatgatccttccgcaggccccctaacgggaaggagggtttattattgaaaaatggaaa

SEQ ID NO: 17 - DNA, 1014 bp, Artificial Sequence
  Primer PP1/27F, reverse sequence
cgaggccgggagtgtgatagagaagcttgcttctcttgatacgttgagcggcggacgggtgagtaacacgtaggcaacctgccctcaagtttgggacaactaccggaaacggtagctaataccgaataattgttttcttcgcctgaaggaaactggaaagacggagcaatctgtcacttggggatgggcctgcggcgcattagctagttggtggggtaacggctcaccaaggcgacgatgcgtagccgacctgagagggtgatcggccacactgggactgagacacggcccagactcctacgggaggcagcagtagggaatcttccgcaatgggcgaaagcctgacggagcaatgccgcgtgagtgatgaaggttttcggatcgtaaagctctgttgccagggaagaacgcttgggagagtaactgctctcaaggtgacggtacctgagaagaaagccccggctaactacgtgccagcagccgcggtaatacgtagggggcaagcgttgtccggaattattgggcgtaaagcgcgcgcaggcggtcatttaagtctggtgtttaatcccggggctcaaccccggatcgcactggaaactgggtgacttgagtgcagaagaggagagtggaattccacgtgtagcggtgaaatgcgtatatatgtggaggaacaccagtggcgaaagcgactctctgggctgtaactgacgcttaagcgcgtaagcgtggggaacaaaaacgattaaataccctgggtttccacaccgtaaacgatgaatgctacgtgtcaaagggttctcccccccttgtgtcgcaaattaaacactttacactccccgcggggagtagagctcccccacggggataccaccgagatttgaggggcccccgccccgttttgaaacttgttgattagtaagacgcccgccaaaaatttataggggggcagcagctaacaaggcggtggggttttcttcttcgccccgaaacaagaaaaaatgtcaccctcgaatt

SEQ ID NO: 18 - DNA, 1063 bp, Artificial Sequence
  Primer PP1/R14192 forward sequence
ttgttcggcggctggctccttgcggttaccccaccgacttcgggtgttataaactctcgtggtgtgacgggcggtgtgtacaagacccgggaacgtattcaccgcggcatgctgatccgcgattactagcaattccgacttcatgcaggcgagttgcagcctgcaatccgaactgagaccggctttgttgggattggctccatctcgcgatttcgcagcccgttgtaccggccattgtagtacgtgtgtagcccaggtcataaggggcatgatgatttgacgtcatccccaccttcctccggtttgtcaccggcagtctatctagagtgcccacccgaagtgctggcaactaaatataagggttgcgctcgttgcgggacttaacccaacatctcacgacacgagctgacgacaaccatgcaccacctgtcttgactgttccgaagaaaaggtacatctctgcaccggtcattcggatgtcaagacctggtaaggttcttcgcgttgcttcgaattaaaccacatactccactgcttgtgcgggtccccgtcaattcctttgagtttcagtcttgcgaccgtactccccaggcggagtgcttaatgtgttaacttcggcaccaagggtatcgaaacccctaacacctagcactcatcgtttacggcgtggactaccagggtatctaatcctgtttgctccccacgctttcgcgcctcagcgtcagttacagcccagagagtcgccttcgccactggtgttcctccacatatctacgcatttcaccgctacacgtggaattccactctcctcttctgcactcaagtcacccagtttccagtgcgatccggggttgagccccgggattaaacaccagacttaaatgaccgcctgcgcgcgctttacgcccaataattccggacaacgcttgccccctacgtattaccgcggctgctggcacgtatttagccggggctttcttctcaggtaccgtcaccttgaaagcagttactctcccaagcgttcttccctggcaacagagctttacgatccgaaaaccttcac

SEQ ID NO: 19 - DNA, 842 bp, Artificial Sequence
  Primer PP2/M1387R, reverse sequence
  misc_feature (793)..(793): n is a, c, g, or t
aggaaaaatgcctaatccccgattttatctctttccgactgcatgcctgctaggcgaaccgtgcaataacgcgtagacacccggcctattgcgagggctccatctcgagatgatcgcttcccctgtagggccattgttgtacgagtgtaacccaggtcagggaggggacgatgatttgacgacctccccgccttcctccggtttgtcaccggcactctatctagactgcccacccgacgtgctggccactaagtataagggttgcgctccttacgcgacttaaccccacatctcacgaaacttgctgactaggccatgccccacctgtctctaccgtcccgaagaaaaaaggcatccctgccccgatctctgttatgtcaagacaaggcaagggtcttctcgtggctctcaaagtgaccgtacctgaccaagaaagcgtggggtaccccgcgcctcctttgaggttaattcttacggcgcaagtccccccccggaagtgttgggtgtgttgccttcgccagcggtggtttaaatatggcgtttaaccccggggctcacccccgatggggatgaaaactgggtgacttgagggtagatgacgagagtgtaattcctcgtggagcgttgaaatgcgtagatatgtctacgaacaccagggtcgacgcgatctatgggctgtaactgacgctgaggagcacaactcttgtggatctgacaggattaaatcccctggtagtccaatccgtagatgatgaccgctgagtgataggggtcttaaatgactgggtggcgnaggtatacattaagcacatacagtaggggaatacgcgtcgcccagaatt

SEQ ID NO: 20 - DNA, 951 bp, Artificial Sequence
  Primer PP1/27F, reverse sequence
ggggaaatgggcggctgctaaacatgcagtcgaacggtgaagcccagcttgctgggtggatcagtggcgaacgggtgagtaacacgtgagcaacctgcccctgactctgggataagcgctggaaacggcgtctaatactggatacgagtagcgaccgcatggtcagttactggaaagatttattggttggggatgggctcgcggcctatcagcttgttggtgaggtaatggctcaccaaggcgtcgacgggtagccggcctgagagggtgaccggccacactgggactgagacacggcccagactcctacgggaggcagcagtggggaatattgcacaatgggcgcaagcctgatgcagcaacgccgcgtgagggatgacggccttcgggttgtaaacctcttttagcagggaagaagcgaaagtgacggtacctgcagaaaaagcgccggctaactacgtgccagcagccgcggtaatacgtagggcgcaagcgttatccggaattattgggcgtaaagagctcgtaggcggtttgtcgcgtctgctgtgaaatccggaggctcaacctccggcctgcagtgggtacgggcagactagagtgcggtaggggagattggaattcctggtgtagcggtggaatgcgcagatatcaggaggaacaccgatggcgaaggcagatctctgggccgtaactgacgcttaggagcgaaagggtggggagcaaacaggcttagataccctggtagtccaccccgtaaacgttgggaactagttgtggggtccattccacggattccgtgacgcagcttacgcattaagttccccgcctggggagtacggccgcaaggctaaaactcaaaggaattgactgggacccgcacaagcggcgaaacatgcggattaattccatgcaactccgaagaaccttaccagggcttgaacatataca

SEQ ID NO: 21 - DNA, 978 bp, Artificial Sequence
  Primer PP1/R14192, forward sequence
ggaatttcgatccaccatatcgacggctccctccacaagggttaggccaccggcttcaggtgttaccgactttcatgacttgacgggcggtgtgtacaagacccgggaacgtattcaccgcagcgttgctgatctgcgattactagcgactccgacttcatgaggtcgagttgcagacctcaatccgaactgggaccggctttttgggattcgctccacctcgcggtattgcagccctttgtaccggccattgtagcatgcgtgaagcccaagacataaggggcatgatgatttgacgtcatccccaccttcctccgagttgaccccggcagtatcccatgagttcccaccattacgtgctggcaacatagaacgagggttgcgctcgttgcgggacttaacccaacatctcacgacacgagctgacgacaaccatgcaccacctgtttacgagtgtccaaagagttgaccatttctggcccgttctcgtatatgtcaagccttggtaaggttcttcgcgttgcatcgaattaatccgcatgctccgccgcttgtgcgggtccccgtcaattcctttgagttttagccttgcggccgtactccccaggcggggaacttaatgcgttagctgcgtcacggaatccgtggaatggaccccacaactagttcccaacgtttacggggtggactaccagggtatctaagcctgtttgctccccaccctttcgctcctcagcgtcagttacggcccagagatctgccttcgccatcggtgttcctcctgatatctgcgcattccaccgctacaccaggaattccaatctcccctaccgcactctagtctgcccgtacccactgcaggccggaggttgagcctccggatttcacagcagacgcgacaaaccgcctacgagctctttacgcccaataattccggaaaacgcttggcgccctacgtattaccgcggctgctggccc

SEQ ID NO: 22 - DNA, 629 bp, Artificial Sequence
  Primer PP1/63F, reverse sequence
  misc_feature (2)..(2): n is a, c, g, or t
  misc_feature (8)..(8): n is a, c, g, or t
  misc_feature (18)..(18): n is a, c, g, or t
  misc_feature (34)..(34): n is a, c, g, or t
gnccgacnagggttgggntgactggctattccangggattcctattcttgaggtcgagttagtaacactcaggtatccttgctattagcttttatgactattggaaacctcgcggaattgcagactattatttattgcaatagtatttggtgggaaaccgaacaatactgggcaagatgatgtgttgtcatcgtcattttcatccgacatctccccggccgtctatcaagagttaccaccattactggctggcaaaatagaacgagagttgcgctcgttgcgggacttaacacaacagctcacgacacgagctgacgacaaccatgcgccacctgtttaaaaatggccctagagttgaaaatttctggtccgttctcataaatgtaaaggcctggtatgttcttaacgttgcgctaaataatccccatgctccgcggattgtgcagggccccgtcgttttccttaatttttagccttgaggacgtactacccaggtggggaactttgtggggtaactgggacgccgaaccccggaatggacgcccaacggcatccaaagtttgtaggggaaattcaagtcatctaaaccggttgatccccacaattctgactaaccgatggcaaggcaa

SEQ ID NO: 23 - DNA, 1307 bp, Artificial Sequence
  Primer PP1/M1387R, forward sequence
gggaacccccaaacggtggctgtctgctatactaggcgacgcctactcatgaggtcgatgtgcagacctcatccgaactgggaccggctttttgggattcgctccacctcgcggtattgcagccctttgtaccggccattgtatcaggcttgaaacccactacataacgggcatgatgatttgacgtcatccccaccttcctccgagttgaccccggcagtatcccatgagttcccaccattacgtgctggcaacatagaacgagggttgcgctcgttgcgggacttaacccaacatctcacgacacgagctgacgacaaccatgcaccacctgtttactagtgtccaaagagttgaccatttctggcccgttctcgtatatgtcaagccttggtaaggttcttcgcgttgcatcgaattaatccgcatgctccgccgcttgtgcgggtccccgtcaattcctttgagttttagccttgcggccgtactccccaggcggggaacttaatgcgttaactgcgtcactgaatccgcggaatggaccccacaactagttcccaacgtttactgggtggactaccaaggtatctaatcctgtttgctccccaccctttcgctcctcaacgtcatttacaggccagatatctgccttcgccatcagtgttcctcctgatatctgggggattccaacgggtactccaggaaattcctaatctccccctaccgaaactctaggtctgtcccgttacccactgccatgccccgaggtttgagtcctcccggactttcacagctagacgtccaccaaaccggcctacgaagctcttttacggcccaaaaaattcccggataatgacttggcgaccctaccctaattacccgcgggctgcctgggcacgatattttattcccggcgccttttttactgcttggttaccggtcactttttggctttcattcacctggctcaaaaaaaggtttaacaaccccgtaaggggccgtccatcccctcaacggcgggcggttggctggcattcaaggccttggcgccccatttggtggcaattaatttccccaacttgcctggccttccccgtcaagcaactcttggggcccgtggttcttcagttcccccaaatgggggggcggggatcaacccttcttcaaaggtcgggcctaaccccaatcctacccccccttagggggagggcgatttgaccgtccacccaaccaaaaccttgaaaaaagggccgggggaaggggcggattcccccccaaccccaaataaaaattcttttttccagggt

(19) United States
(12) Patent Application Publication
(10) Pub. No.: US 2025/0282811 A1
(43) Pub. Date: Sep. 11, 2025

(54) A 3-O-MONODESMOSIDIC SAPONINS EXTRACT, STABLE AT PHYSIOLOGICAL PH, METHOD FOR PREPARING IT FROM A QUILLAJA PLANT EXTRACT CULTURED WITH A MICROBIAL CONSORTIUM, USES AND SUCH MICROBIAL CONSORTIUM

(71) Applicants: DESERT KING CHILE S.A, Quilpué, Valparaíso (CL); PONTIFICIA UNIVERSIDAD CATÓLICA DE VALPARAÍSO, Valparaíso (CL)

(72) Inventors: Leandro PADILLA IGLESIAS, Quilpué, Valparaíso (CL); Leda Marcela GUZMAN MALUENDA, Curauma (CL)

(21) Appl. No.: 18/697,798

(22) PCT Filed: Oct. 2, 2021
(86) PCT No.: PCT/IB2021/059065

§ 371 Date: Apr. 2, 2024

(51) Int. Cl. C07H 15/256

(52) U.S. Cl. CPC C07H 15/256

(57) ABSTRACT

The present invention relates to a biotransformation process effected by cultures of a microbial consortium for the preparation of a Quillaja plant extract with antibacterial and cytotoxic properties, comprising one or more monodesmosidic 3-O-glycosides of quillaic acid of formula (I),

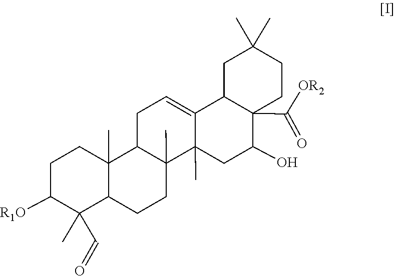

wherein R₁ is {xylopyranosyl-(1→3)-glucopyranosiduronic acid} or a salt thereof, {xylopyranosyl-(1→3)-glucopyranosiduronate salt}; {rhamnopyranosyl-(1→3)-glucopyranosiduronic acid} or a salt thereof, {rhamnopyranosyl-(1→3)-glucopyranosiduronate salt}; or {galactopyranosyl-(1→2)-glucopyranosiduronic acid} or a salt thereof, {galactopyranosyl-(1→2)-glucopyranosiduronate salt}; or {glucopyranosiduronic acid} or a salt thereof, {glucopyranosiduronate salt}, and R₂ is hydrogen or a cation specie.

Such microbial consortium comprising microorganisms selected from Rhodotorula sp having accession number RGM 3031 (Colony P) dated Jul. 22, 2020 of the Chilean Collection of Microbial Genetic Resources (CChRGM); Microbacterium sp having accession number RGM 3021 (Colony Y) dated Aug. 7, 2020 of the Chilean Collection of Microbial Genetic Resources (CChRGM); Mucor sp having accession number RGM 2984 (Colony C) dated Aug. 7, 2020 of the Chilean Collection of Microbial Genetic Resources (CChRGM); Paenibacillus sp and having accession number RGM 3032 (Colony W) dated Sep. 3, 2020 of the Chilean Collection of Microbial Genetic Resources (CChRGM). The present invention is also related to the extract (SPH03) obtained from the above-described method, uses and such microbial consortium. The present extract is stable at physiological pH and has a strong reduced hemolytic effect, a cytotoxic effect on cancer cells and an antibacterial effect on oral pathogens such as Streptococcus mutans.

### AN INCORPORATION BY REFERENCE STATEMENT REGARDING THE MATERIAL IN

An XT.25 file for a “Sequence Listing” (see § 1.831(a), submitted via the USPTO patent electronic filing system, identifying
the name of the file: Sequence LISTING REVISED;
the date of creation of each file: Apr. 10, 2025
and the size of each file in bytes 22 kb bytes.

### BACKGROUND OF THE INVENTION

### Field

The present invention relates to a biotransformation process effected by cultures of a microbial consortium for the preparation of monodesmosidic 3-O-glycosides of quillaic acid, also referred as 3-O-monodesmosidic Quillaja saponins. Such microbial consortium comprising microorganisms selected from Rhodotorula sp having accession number RGM 3031 dated Jul. 22, 2020 (Colony P) of the Chilean Collection of Microbial Genetic Resources (CChRGM); Microbacterium sp having accession number RGM 3021 dated Aug. 7, 2020 (Colony Y) of the Chilean Collection of Microbial Genetic Resources (CChRGM); Mucor sp having accession number RGM 2984 dated Aug. 7, 2020 (Colony C) of the Chilean Collection of Microbial Genetic Resources (CChRGM); Paenibacillus sp and having accession number RGM 3032 dated Sep. 3, 2020 (Colony W) of the Chilean Collection of Microbial Genetic Resources (CChRGM). In particular, the method of this invention provides an extract of 3-O-monodesmosidic saponins prepared from a partially hydrolized Quillaja plant extract cultured with the above-described microbial consortium.

### Description of Related Art

The biotransformation of saponins has been widely studied and documented for the ginsenosides, the bidesmosidic triterpenic saponins of Panax ginseng (ginseng). Panax ginseng accumulates ginsenosides (Ru et al., Drug Discovery and Therapeutics. 9: 23-32 (2015); Shin et al., Journal of Ginseng Research. 39: 287-298 (2015)), mostly the compounds Rb1, Rb2, Rc, Rd, Re and Rg1 accounting more than 99% of the saponin composition of ginseng (Cheng et al., Journal of Microbiology and Biotechnology. 17: 1937-1943 (2007); Kim et al., Journal of Ginseng Research. 36: 291-297 (2012)). However, some minor ginsenosides scarcely found in the extracts of Panax ginseng have outstanding properties, as for example: i) the ginsenoside M1 (also known as Compound K) has antitumoral, antidiabetic, hepatoprotective and antioxidant properties (Kim et al., 2012; Li et al., Molecules. 16: 10093-10103 (2011)); ii) the ginsenoside F2 has antitumoral properties and is also a promising agent to prevent hair-loss (Shin et al., Journal of Ginseng Research. 35: 86-92 (2012); Shin et al., Biological and Pharmaceutical Bulletin. 37: 755-763 (2014)), and iii) the compound GP-17 (also known as gipenoside XVII) has potential as neuroprotective agent against the Alzheimer's disease (Meng et al., Journal of Alzheimers Disease. 52:1135-1150 (2016)).

In order to make available large amounts of ginsenoside M1, ginsenoside F2 and the compound GP-17, a number of biotechnological process based in enzymatic conversion and microbial transformation have been described. Li et al., (2011) developed the preparation of the ginsenoside M1 from a mixture of ginsenosides comprising the ginsenosides Rb1, Rb2, Rc y Rd), catalyzed by the enzyme snailase. Cheng et al., (2007) described the preparation of the ginsenoside F and the compound GP-17 by biotransformation of the ginsenoside Rb1 in liquid cultures of the bacterium Intrasporangium sp. GS603. Wang et al., (Molecules. 20: 19291-19309 (2015)) described the preparation of the ginsenoside M1 by biotransformation of the ginsenoside Rb1 in liquid cultures of the fungus Cordyceps sinensis.

The conversion of saponins by biotechnological methods has been even extended to the preparation of novel compounds from the ginsenosides of Panax ginseng. Ko et al., (Bioscience, Biotechnology and Biochemistry. 65: 1223-1226 (2001)) described the synthesis of two novel ginsenosides by an enzyme catalyzed reaction between the ginsenoside Re and the glycosyl donors p-nitrophenyl-β-D-xylopyranoside and phenyl-β-D-xilopyranoside. Han et al., (Molecules. 15: 399-406 (2010)) prepared the novel antitumoral ginsenoside ORh1 from a mixture comprising ginsenosides Rg1 and Re by a two-step procedure combining enzymatic hydrolysis and chemical acylation.

Nor of the previously enumerated approaches for bioconversion of saponins has been described for the triterpenic saponins of Quillaja saponaria Molina.

The extracts of the bark and pruned branches of Quillaja saponaria Molina contain triterpenic saponins (Resnik, Chemical and Technical Assessment 61st JECFA. FAO (2004)), bidesmosidic glycosides of quillaic acid with two oligosaccharide chains attached to C-3 and C-28 positions (Guo et al., Phytochemistry. 48: 175-180 (1998)). The four most abundant saponins in commercial extracts of Quillaja saponaria Molina are the bidesmosidic glycosides QS-7, QS-17, QS-18 and QS-21 (Kensil et al., Journal of Immunology. 146: 431-437 (1991)).

The immune-stimulant activity of extracts of triterpenic saponins of Quillaja saponaria Molina was first described in the 1930s, and later used to improve a foot-and-mouth disease vaccine. In 1978, Dalsgaard first obtained an enriched mixture of saponins (Quil A) from this extract and found Quil A stimulated both humoral and cellular immunity as well as to induce differential antibody isotypes (Sun et al., Vaccine. 27: 1787-1796 (2009)). Since then, the production of several partially purified saponin extracts has been described, for example, SuperSap (Natural Response) and the chromatographically isolated fractions QH-A and QH-C(Rönnberg, et al. Vaccine. 13: 1375-1382 (1995)).

In addition to the immune-stimulant properties, other biological effects have been associated to the triterpenic saponins of Quillaja saponaria Molina. The US Patent Application US 2007 0264401 A1, discloses the use of Quillaja extracts as beverage preservatives against microbial spoilage by some mold yeast and bacteria. For example, the addition of 100 ppm of Quillaja extracts to sucrose solutions at pH 3.0 stored at 25° C. prevent the growth of the bacteria Gluconacetobacter xylinus, Alicyclobacillus acidoterrestris, Bacillus stearothermophilus.

The US Patent Application US 2005 0175623 A1 discloses the in vitro killing and inhibition effects of a commercial extract of Quillaja saponins and some sub-fractions prepared from the said commercial extract on a range of tumor cell lines comprising Hep-G2, HuH-7, HT-29, asPC-1, renal carcinoma cell line, MCF-7, TSU, DMS-53, H-157, Jurkat, and Sp2/0-Ag14. Some of the said sub-fractions of Quillaja saponins, alone and in combination with other anti-cancer therapeutic agents showed inhibition of cancer cell growth or proliferation through deconstruction of the cell membrane, and cell cycle arrest at G1, as well as promoting induction of specific cancer apoptosis. The inhibitory effect of saponins observed with cancer cells was not obvious in normal cells such as white blood cells, human hepatocytes, 3T3 mouse cell line and mouse fibroblasts. However, some of the fractions mentioned were shown to be toxic in vivo to mice upon peritoneal and muscular administration. For example, the sub-fraction QSF-IV was also highly toxic to liver (10 mg QSF-IV per Kg of body weight caused 50% death in mice). Although a dose of 3 QSF-IV per Kg of body weight could be injected repeatedly without lethal effects, slight liver damage was still observed. The latter results were consistent with the adverse effects associated with intradermal administration of Quillaja saponins to mice, such as hemolysis and lethality (Kensil et al., 1991). To prevent the adverse effects described, as well as to increase the chemical stability of saponins at physiological pH in anticancer applications, the use of liposomes and nanoparticle formulations has been proposed. For example, the US Patent Application US 2010 0119591 A1 discloses the is use of lipid containing particles referred as KGI particles comprising cholesterol, phosphatidyl-cholin and Quillaja saponin fraction QH-C, as killing agents against malign monoblast cells U937. In addition to their cell killing properties, KGI particles are significantly less hemolytic than free QH-C saponins. According to the related US Patent Application US 2014 0234349 A1, the cell killing effects against U937 cells of nanoparticles G3 comprising only cholesterol and Quillaja saponin fraction QH-C, are comparable to those exerted by KGI particles.

Neither QuilA nor SuperSap are suitable as ingredient of pharmaceutical formulations for human use because of the chemical heterogeneity of the saponin composition, which includes more than 50 different saponins. This chemical heterogeneity has led to the development of procedures for the chromatographic isolation of purified saponins (Kensil & Marciani. U.S. Pat. No. 5,057,540 (1991); Kernsten et al. Canadian Patent 2, 094,600 (1998)). Practical and economic constraints limit the commercial production of purified saponins of Quillaja saponaria Molina, and uses thereof, to the most abundant bidesmosidic saponins, for example QS-7, QS-17, QS-18 and QS-21.

As previously mentioned, intradermal administration of some bidesmosidic Quillaja saponins may cause side effects, because of their hydrophobic-lytic properties resulting in trapping at the site of administration, causing cell and tissue destruction, leading to local and systemic adverse reactions (Hu et al., International Journal of Nanomedicine. 5: 51-62 (2010)). These adverse effects have prevented the widespread use of saponins for injectable administration in therapeutic applications.

An additional major concern related to the use of bidesmosidic saponins of Quillaja saponaria Molina is the lack of chemical stability at physiological pH, leading to the hydrolysis of the ester bond joining the substituent at C-28 position (Pillion et al. Journal of Pharmaceutical Sciences. 85: 518-524 (1996)).

Thus, there is an unmet need of stable Quillaja saponins lacking the above adverse effects. The monodesmosidic Quillaja saponins lacking the substituents at C-28 are scarcely found in Quillaja saponaria Molina (Guo et al., Phytochemistry. 48: 175-180 (1998)). Guo et al., 1998 purified from the QH-A fraction of extracts of Quillaja saponaria three monodesmosidic 3-O-glycosides of quillaic acid, chemically characterized as quillaic is acid 3-O-{xylopyranosyl-(1→3)-[galactopyranosyl-(1→2)]-glucopyranosiduronic acid}

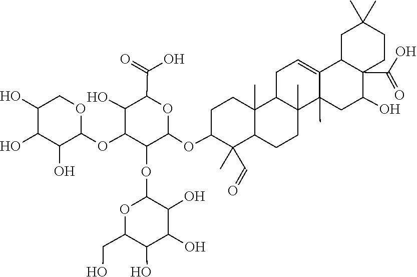

hereinafter referred as Compound I, quillaic acid 3-O-{rhamnopyranosyl-(1→3)-[galactopyranosyl-(1→2)]-glucopyranosiduronic acid}

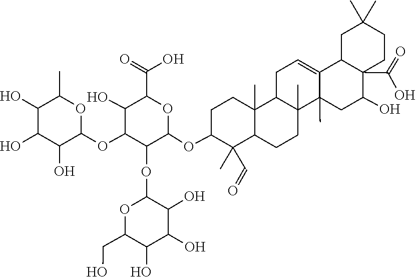

hereinafter referred as Compound II and quillaic acid 3-O-{rhamnopyranosyl-(1→2)-glucopyranosiduronic acid}

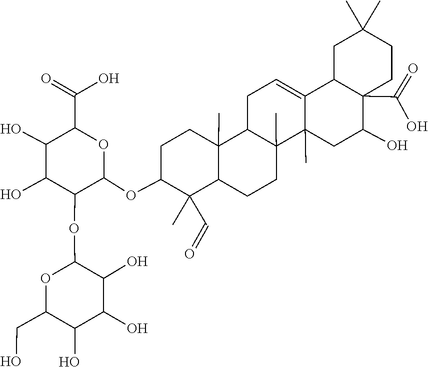

hereinafter referred as Compound III.

According to the data of Guo et al., 1998 the yields of the recovery of the Compounds I, II and III from QH-A fraction by chromatographic means were 2.24%, 0.71%, and 0.29%, respectively.

The U.S. Pat. No. 5,650,398 discloses a method to produce the compound QH-957 having the same structure of the Compound I, by heating a QS-21 solution in phosphate-buffered saline at pH 6-7 for 48-72 hours at 100°−105° C., followed by purification of Compound I by reverse-phase HPLC in a water/acetonitrile gradient, containing 0.15% trifluoroacetic acid, and lyophilization of the corresponding fractions.

According to Van Setten et al., (Rapid Communications in Mass Spectrometry. 9: 660-666 (1995)) the base catalyzed hydrolysis of the partially purified extract of QuilA with KOH in 50% ethanol at reflux temperature for 7 hours, yielded only two main products with molecular masses 956 and 970 D, consistent with the molecular masses of the anionic forms of Compounds I and II, respectively; the production of the Compound III under these conditions was not detected.

Moreover, the U.S. Pat. No. 5,817,314 discloses the purification by chromatographic means from extracts of the bark of Quillaja saponaria Molina, of a compound QS-L1 having the same molecular mass of the anionic form of Compound I, which possess immune-stimulant properties and no hemolytic activity in the range 4-500 μg/ml. However, the isolation of the compound QS-L1 described in the latter disclosure still depends on the supply of bark extracts to secure the isolation of the compound QS-L1. Thus, there is an unmet need to find novel methods to produce 3-O-monodesmosidic saponins of Quillaja saponaria Molina suitable for pharmaceutical formulations in greater yield.

### SUMMARY OF THE INVENTION

An object of the present invention is to provide an extract of saponin compounds, hereafter designated as extract SPH03, comprising one or more 3-O-monodesmosidic saponins of Formula (I),

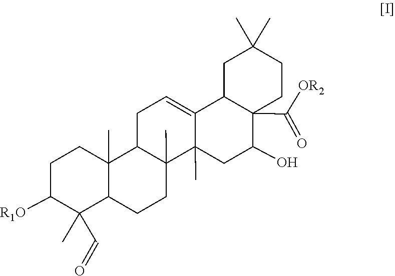

wherein R₁ is {xylopyranosyl-(1→3)-glucopyranosiduronic acid} or a salt thereof, including {xylopyranosyl-(1→3)-glucopyranosiduronate salt}; {rhamnopyranosyl-(1→3)-glucopyranosiduronic acid} or a salt thereof, including {rhamnopyranosyl-(1→3)-glucopyranosiduronate salt}; or {galactopyranosyl-(1→2)-glucopyranosiduronic acid} or a salt thereof, including {galactopyranosyl-(1→2)-glucopyranosiduronate salt}; or {glucopyranosiduronic acid} or a salt thereof, {glucopyranosiduronate salt}, and R₂ is hydrogen or a cation specie.

The 3-O-monodesmosidic saponins of Formula [I](as described above) in the extract SPH03 are stable at physiological pH, have no or little hemolytic effect, i.e., a strong reduce hemolytic effect, and exhibit, cytotoxic effect on cancer cells and antibacterial effect on oral pathogens.

Another object of the present invention is to provide a novel microbial consortium [HNR2] for the microbial biotransformation of Quillaja plant extract comprising Compounds I and II, to the extract SPH03. Such microbial consortium comprising microorganisms selected from Rhodotorula sp having accession number RGM 3031 (Colony P) dated Jul. 22, 2020 of the Chilean Collection of Microbial Genetic Resources (CChRGM); Microbacterium sp having accession number RGM 3021 (Colony Y) dated Aug. 7, 2020 of the Chilean Collection of Microbial Genetic Resources (CChRGM); Mucor sp having accession number RGM 2984 (Colony C) dated Aug. 7, 2020 of the Chilean Collection of Microbial Genetic Resources (CChRGM); Paenibacillus sp and having accession number RGM 3032 (Colony W) dated Sep. 3, 2020 of the Chilean Collection of Microbial Genetic Resources (CChRGM).

Still another object of this invention is to provide a biotransformation process to overcome the limitations for production of large amounts of the extract SPH03 substantially free of bidesmosidic saponins of Quillaja plants, preferably Quillaja saponaria Molina extract, based on liquid fermentation cultures of HNR2 microbial consortium catalyzing the conversion of an extract comprising Compounds I and II, to the extract SPH03, wherein such microbial consortium is as mentioned above.

A further object of this invention is to provide a formulation of the liquid broth employed for the biotransformation of an extract comprising Compounds I and II, to the extract SPH03 catalyzed by HNR2, where the productivity of the process is increased.

A still further object of this invention is to provide a method to isolate the extract SPH03 from the fermentation broth employed in the biotransformation.

These objects are accomplished by the herein described invention which comprises steps of hydrolysis of aqueous extracts of Quillaja plant extract, preferably, a Quillaja saponaria Molina extract, followed by the biotransformation of the resulting hydrolyzed extract comprising Compounds I and II by a HNR2 microbial consortium culture as mentioned above, and the subsequent recovery of the extract SPH03 substantially free of bidesmosidic saponins of Quillaja saponaria Molina from the fermentation broth by liquid extraction or chromatographic means. The method of the present invention allows the practical, reliable and sustainable production of large amounts of the extract SPH03 as well as the 3-O-monodesmosidic saponins of Formula (I).

This invention also provides examples of the use of the extract SPH03 as well as its monodesmosidic saponins of Formula (I) as cytotoxic and antibacterial agents.

### BRIEF DESCRIPTION OF THE DRAWINGS

FIG. 1: RP-UHPLC trace of VaxSap® (VS, highly purified powder extract, mainly containing triterpene saponins ca. 90% w/w), a spray-dried and highly purified extract of saponins of Quillaja saponaria Molina.

FIG. 2: The conversion of Quillaja saponaria saponins (VaxSap®) to a mixture comprising Compounds I and II, as verified by RP-UHPLC chromatography.

FIG. 3: TLC analysis of the eluate fractions after the hydrolysis of Quillaja saponaria saponins (VaxSap®).

FIG. 4: Gel like substance floating at the surface of a spoiled aqueous extract of Quillaja saponaria Molina stored at room temperature, employed as the source for the isolation of the consortium HNR2 (as defined above).

FIG. 5: Four major types of colonies (Mucor sp (RGM 2984, C), Rhodotorula sp (RGM 3031, P), Paenibacillus sp (RGM 3032, W) and Microbacterium sp (RGM 3021, Y) found upon plating of inoculum HNR2 on Luria Bertani Agar (LBA).

FIG. 6A: Agarose electrophoresis of PCR products obtained from genomic DNA of colonies C, P, W and Y, with primer pairs PP1 (SEQ ID Nos.: 1 and 2).

FIG. 6B: Agarose electrophoresis of PCR products obtained from genomic DNA of colonies C, P, W and Y, with primer pairs PP2 (SEQ ID Nos.: 3 and 4).

FIG. 6C: Agarose electrophoresis of PCR products obtained from genomic DNA of colonies C, P, W and Y, with primer pairs PP3 (SEQ ID Nos.: 5 and 6).

FIG. 6D: Agarose electrophoresis of PCR products obtained from genomic DNA of colonies C, P, W and Y, with primer pairs PP4 (SEQ ID Nos.: 7 and 8).

FIG. 7: RP UHPLC analysis of supernatants taken during the biotransformation of Compounds I and II to SPH03 catalyzed by consortium HNR2 as defined above.

FIG. 8: TLC analysis of supernatants taken during the biotransformation of Compounds I and II to SPH03 catalyzed by consortium HNR2 as defined above.

FIG. 9: TLC analysis of supernatants taken during the biotransformation of Compounds I and II to SPH03 catalyzed by consortium HNR2 as defined above, showing the effect of the broth formulation on the production of SPH03.

FIG. 10: Effect of xylan concentrations and Compounds I and II on the conversion of an extract containing Compounds I and II in SPH03 by liquid cultures of consortium HNR2 as defined above.

FIG. 11: RP UHPLC chromatogram of the SPH03 product purified by preparative RP HPLC.

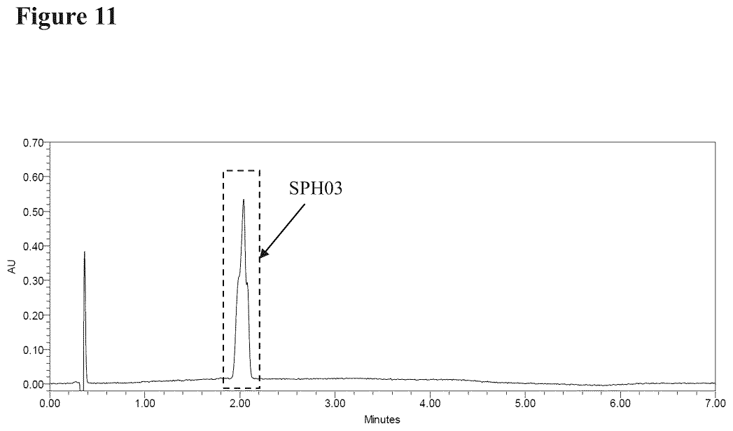

FIG. 12: Mass spectrum of the Compound III of the extract SPH03, obtained by high performance liquid chromatography coupled to electrospray ionization time of flight mass spectrometry analysis (HPLC-ESI-TOF-MS).

FIG. 13: Mass spectrum of the Compound IV of the extract SPH03, obtained by HPLC-ESI-TOF-MS analysis.

FIG. 14: Mass spectrum of the Compound V of the extract SPH03, obtained by HPLC-ESI-TOF-MS analysis.

FIG. 15: Mass spectrum of the Compound VI of the extract SPH03, obtained by HPLC-ESI-TOF-MS analysis.

FIG. 16A: Hemolytic effect on chicken red blood cells (CRBC) of Quillaja saponins (VetSap, Veterinary Vaccine Adjuvant from a semi-purified Saponin Fraction of the Bark of the Quillaja Saponaria tree), an extract of compounds I/III and SPH03.

FIG. 16B: Hemolytic effect on chicken red blood cells (CRBC) of an extract of compounds I/III and SPH03.

FIG. 17: UHPLC analysis of samples of 0.5 g/L solutions of Quillaja saponins and SPH03 in Mc Ilvine buffer (pH 7.4) incubated at 37° C.

FIG. 18A: Effect of the extract comprising Compounds I and II on cell viability tumoral cells (AGS, SNU1 and GES-1 cells cultured in vitro, as determined by MTS assay, a colorimetric cell proliferation assay.

FIG. 18B: Effect of SPH03 on cell viability tumoral cells (AGS, SNU1) and GES-1 cells cultured in vitro, as determined by MTS assay.

FIG. 19: Muse™ Annexin V and Dead Cell apoptosis assay applied to AGS cells untreated, treated with Cisplatin 55 microM, SPH03 50 microM and SPH03 70 microM.

FIG. 20: Muse™ Annexin V and Dead Cell apoptosis assay applied to SNU1 cells untreated, treated with Cisplatin 55 microM, SPH03 50 microM and SPH03 70 microM.

FIG. 21: Inhibition of biofilm formation by Streptococcus mutans UA159 and Enterecoccus faecalis ATCC 29212 grown in BHIB broth supplemented with SPH03.

### DETAILED DESCRIPTION

As described above, the present application relates to a method for obtaining an Quillaja plant extract comprising monodesmosidic 3-O-glycosydes of quillaic acid, being stable at physiological pH, have no or little hemolytic effect, i. e., a strong reduced hemolytic effect, and exhibit, cytotoxic effect on cancer cells and antibacterial effect on oral pathogens such as Streptococcus mutans.

Referring now to the invention in more detail, the extract SPH03, comprising one or more of the monodesmosidic saponins of Formula (I), herein below referred as Compounds III (as described above), IV, V and VI, are prepared by biotransformation of a plant biomass extract comprising glycosides of quillaic acid with liquid fermentation cultures of HNR2 microbial consortium, being compounds HI, IV, V and VI as follows:

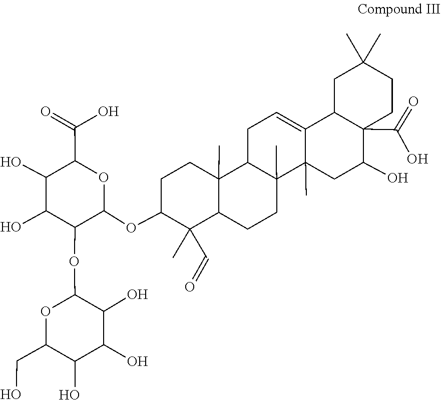

Compound III: Quillaic acid 3-O-{galactopyranosyl-(1→2)-glucopyranosiduronic acid} or a salt thereof, quillaic acid 3-O-{galactopyranosyl-(1→2)-glucopyranosiduronate salt} and R₂ is hydrogen or a cation specie.

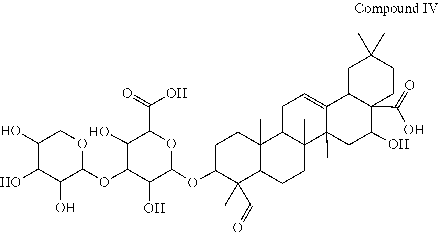

Compound IV: Quillaic acid 3-O-{xylopyranosyl-(1→3)-glucopyranosiduronic acid} or a salt thereof, quillaic acid 3-O-{xylopyranosyl-(1→3)-glucopyranosiduronate salt} and R₂ is hydrogen or a cation specie.

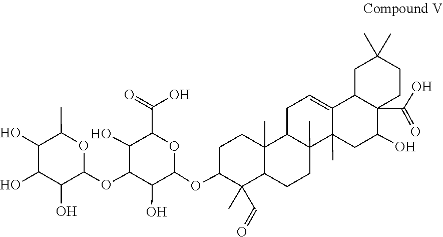

Compound V: Quillaic acid 3-O-{rhamnopyranosyl-(1→3)-glucopyranosiduronic acid} or a salt thereof, quillaic acid 3-O-{rhamnopyranosyl-(1→3)-glucopyranosiduronate salt} and R₂ is hydrogen or a cation specie.

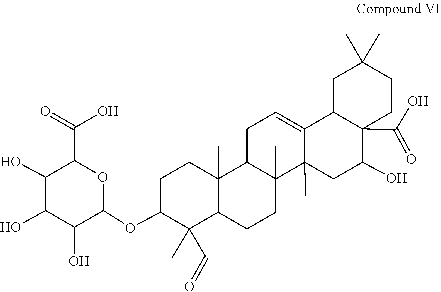

Compound VI: Quillaic acid 3-O-glucopyranosiduronic acid or a salt thereof, quillaic acid 3-O-glucopyranosiduronate salt and Rz is hydrogen or a cation specie.

Such microbial consortium was taken out from spoiled quillaja extracts. Table 1 below summarizes the characteristics of the microorganisms forming the consortium as well as its deposit-related information

TABLE 1

| | Name of the | Characteristics of the | Deposit No |
|---|---|---|---|
| No | microorganism. | microorganism. | in CChRGM |
| 1 | Paenibacillus | Facultative anaerobic, endospore- | RGM 3032 |
| | sp. (W) | forming bacteria. | Sep. 3, 2020 |
| 2 | Microbacterium | Microbacterium is a genus of | RGM 3021 |
| | sp. (Y) | bacteria in the family | Aug. 7, 2020 |
| | | Microbacteriaceae. | |
| 3 | Rhodotorula | Common environmental | RGM 3031 |
| | sp. (P) | basidiomycetous yeasts, which can | Jul. 22, 2020 |
| | | be found in soil, ocean and lake | |
| | | water, fruit juice and milk. | |
| 4 | Mucor sp. (C) | Mucor is a microbial genus of | RGM 2984 |
| | | approximately 40 species of molds | Aug. 7, 2020 |
| | | commonly found in soil, digestive | |
| | | systems, plant surfaces, foods and | |
| | | rotten vegetable matter. | |

The extract SPH03, comprising one or more of the monodesmosidic saponins of Formula (I) as defined above, which are referred below as Compounds III, IV, V and VI, being stable at physiological pH, having no or little hemolytic effect, i.e, a strong reduced hemolytic effect, and exhibit anti-tumor effect on cancer cells and antibacterial effect on oral pathogens, such as Streptococcus mutans.

Thus, the term “extract SPH03” is used herein below to refer an extract of saponin compounds comprising one or more 3-O-monodesmosidic saponins of Formula (I),

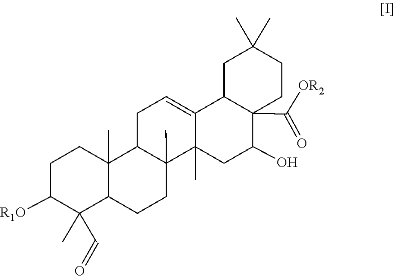

wherein R₁ is {xylopyranosyl-(1→3)-glucopyranosiduronic acid} or a salt thereof, including {xylopyranosyl-(1→3)-glucopyranosiduronate salt}; {rhamnopyranosyl-(1→3)-glucopyranosiduronic acid} or a salt thereof, including {rhamnopyranosyl-(1→3)-glucopyranosiduronate salt}; or {galactopyranosyl-(1→2)-glucopyranosiduronic acid} or a salt thereof, including {galactopyranosyl-(1→2)-glucopyranosiduronate salt}; or {glucopyranosiduronic acid} or a salt thereof, {glucopyranosiduronate salt}, and R₂ is hydrogen or a cation specie, which is substantially free of bidesmosidic saponins.

The term “HNR2 microbial consortium” is used herein to refer a microbial consortium comprising microorganisms selected from Rhodotorula sp having accession number RGM 3031 (colony P) dated Jul. 22, 2020 of the Chilean Collection of Microbial Genetic Resources (CChRGM); Microbacterium sp having accession number RGM 3021 (colony Y) dated Aug. 7, 2020 of the Chilean Collection of Microbial Genetic Resources (CChRGM); Mucor sp (colony C) having accession number RGM 2984 dated Aug. 7, 2020 of the Chilean Collection of Microbial Genetic Resources (CChRGM); Paenibacillus sp and having accession number RGM 3032 (colony W) dated Sep. 3, 2020 of the Chilean Collection of Microbial Genetic Resources (CChRGM).

The term “hemolytic effect” as used herein refers to the lysis of erythrocyte cells.

The term “cytotoxic effect” as used herein refers to the inhibition of the replication of cancer cells and the decrease in the number of cancerous cells in vitro or in vivo.

The term “antibacterial effect” as used herein refers to the inhibition of the replication of bacterial cells and the decrease in the number of bacterial cells in vitro or in vivo.

The term “plant biomass” as used herein refers to as used herein, refers to any biological material originated from the kingdom Plantae. For example, the biomass can be the bark, trunk, leaves, stems, roots, seeds, flowers, fruits or a combination thereof.

The term “plants containing glycosides of quillaic acid” as used herein refers, but not limited to, the following plant species: Quillaja saponaria Molina, Quillaja brasiliensis and Saponaria vaccaria.

The present invention provides a method for production of the extract SPH03 substantially free of bidesmosidic saponins as defined above, comprising the following steps:
a) Preparing a partially hydrolyzed extract from a plant biomass extract containing glycosides of quillaic acid and obtaining a crude hydrolysate,
b) Recovering from the crude hydrolysate obtained in step a), an extract of monodesmosidic saponins comprising Compounds I and II,
c) Culturing such extract obtained in step b) into a liquid fermentation culture of a microbial consortium (HNR2), wherein such microbial consortium (HNR2) comprising microorganisms selected from Rhodotorula sp having accession number RGM 3031 (colony P) dated Jul. 22, 2020 of the Chilean Collection of Microbial Genetic Resources (CChRGM); Microbacterium sp having accession number RGM 3021 (colony Y) dated Aug. 7, 2020 of the Chilean Collection of Microbial Genetic Resources (CChRGM); Mucor sp having accession number RGM 2984 (colony C) dated Aug. 7, 2020 of the Chilean Collection of Microbial Genetic Resources (CChRGM); Paenibacillus sp and having accession number RGM 3032 (colony W) dated Sep. 3, 2020 of the Chilean Collection of Microbial Genetic Resources (CChRGM), and
d) Removing such microbial consortium (HNR2) from such fermentation broth obtained in step c) and recovering an extract substantially free of microorganisms and bidesmosidic saponins.

In preferred embodiments, the plant biomass for the preparation of the extract SPH03 is selected from bark, pruned branches or leaves of a Quillaja plant, preferably, from Quillaja saponaria Molina.

Such extract of biomass can be preferably hydrolyzed in basic media, with an inorganic or organic base, prior to transform the bidesmosidic saponins in 3-O-monodesmosidic saponins (Compounds I and II).

In a preferred embodiment, the extract of biomass of Quillaja saponaria Molina can be preferably hydrolyzed in KOH solutions and then also submitted to aqueous ethanol to render a crude hydrolysate comprising Compounds I and II as well as other impurities.

Such crude hydrolysate can be also fractionated rendering an enriched extract comprising Compounds I and II. In a preferred embodiment the crude hydrolysate is fractionated by chromatography on a hydrophobic resin.

Such enriched extract comprising Compounds I and II can be also added to the culture broth inoculated with a consortium of biotransforming microorganisms. Such microbial consortium can also comprise one or more of Paenibacillus amylolyticus, Paenibacillus tundra, Paenibacillus tylopili, Microbacterium hydrocarbonoxydans, Microbacterium luteolum, Microbacterium phyllosphaerae, Rhodotorula mucilaginosa, Mucor rcemosus, Cylindrocarpon spp., Aspergillus niger and Aspergillus parasiticus. Preferably, such consortium is consortium HNR2 as defined above.

The culture broth supplemented with the enriched extract comprising Compounds I and II and inoculated with the consortium of biotransforming microorganisms (HNR2) is cultivated rendering the biotransformed fraction SPH03 comprising the Compounds III, IV, V and VI. To improve the production of SPH03 by the consortium of biotransforming microorganisms, the culture broth can be further supplemented with oligosaccharides of xylose.

The extract SPH03 containing Compounds IIII, IV, V and VI may be isolated from the biotransformation broth by solvent extraction followed by solvent evaporation at reduced pressure, or by chromatography followed by solvent removal at reduced pressure and lyophilization.

Compounds HI, IV, V and VI of the extract SPH03, may be identified by a pseudo-molecular ion of the monosodium adducts when analyzed by mass spectrometry using electrospray ionization (ESI-MS).

The advantages of the present invention include, without limitation, the production of the extract SPH03, substantially free of bidesmosidic saponins. Other advantage is the production of the extract SPH03 of a range of plant biomass. The present method may be further understood by reference to the following Examples, which are not intended to be limiting of the scope of the invention.

### EXAMPLES

### Example 1: Analysis of Saponin Fractions by UHPLC Liquid Chromatography

The chemical profile of the saponins present in substrate and product solutions of biotransformation steps was determined by ultrahigh performance liquid chromatography (UHPLC) in a Waters Acquity H-Class UPLC chromatography system coupled to a Waters PDA Acquity diode array absorbance detector. The analytical column used was an Acquity UPLC® BEH C-18 (particle size 1.7 μm, inner diameter 2.1 mm; length 50 mm) (Waters, MA, USA). The column was maintained at 30° C. during each chromatographic run. The injected samples were eluted at constant flow rate (0.41 ml/min) in gradient elution mode employing the following mobile phases: Solvent A: Formic acid 0.15% v/v in water, and; Solvent B: Formic acid 0.15% v/v in acetonitrile. The solvent gradient employed is described as follows: Step 1: 0.0-2.5 min from 34% to 45% of Solvent B; Step 2: 2.5-3.5 min 45% of Solvent B; Step 3: 3.5-5.0 min from 45% to 34% of Solvent B, and; Step 4: 5-0-7.0 min 34% of Solvent B. The eluted peaks were detected by monitoring of the absorbance at 210 nm.

### Example 2: Characterization of the Products of Biotransformation by Liquid Chromatography Coupled to Mass Spectrometry

The triterpenes present in the biotransformation product were characterized by high performance liquid chromatographic methods using “time of flight” mass detection (HPLC-ESI-TOF-MS) in an Agilent HPLC 1200 HPLC chromatography system coupled to an Agilent Q-TOF 6520 mass spectrometer detector. The column used was a Luna® 5 μC18 (particle size 5.0 μm, inner diameter 4.6 mm; length 250 mm) (Phenomenex, CA, USA). The column was maintained at 30° C. during each run. The injected samples were eluted in gradient elution mode at constant flow rate (1.0 ml/min) employing the following mobile phases: Solvent A: Formic acid 0.10% v/v in water, and; Solvent B: Formic acid 0.10% v/v in acetonitrile. The solvent gradient employed is described as follows: Step 1: 0-30 min from 34% to 45% of Solvent B; Step 2: 30-35 min 45% of Solvent B; Step 3: 35-36 min from 45% to 34% of Solvent B, and; Step 4: 36-50 min 34% of Solvent B.

The eluted compounds were ionized in electrospray positive ionization mode. The mass detector parameters were as follows: Capillary potential: 4.0 kV; fragmentor voltage: 150 V, and; mass range: 100-3000 Th.

### Example 3: Analysis of Saponin Fractions by Thin-Layer Chromatography (TLC)

In addition to liquid chromatography, thin-layer chromatography (TLC) was employed to characterize the composition of substrates and biotransformation products. To this end 2-4 microliters of sample (25 g/L) were loaded onto aluminum TLC plates covered with Silicagel 60. The plate was developed with a mixture 30:16:8:1 of ethyl acetate/ethanol/water/acetic acid. After this procedure, the plate was air-dried and sprayed with a visualization reagent prepared by mixing of 48 ml ethanol, 2 ml deionized water, 2.24 ml p-anisaldehyde and 1.35 ml concentrated sulfuric acid. The sprayed plate was heated 10 min at 105° C. to visualize the bands.

### Example 4: Preparation of an Extract Containing Compounds I and II, Employed as Substrate of the Biotransformation Process

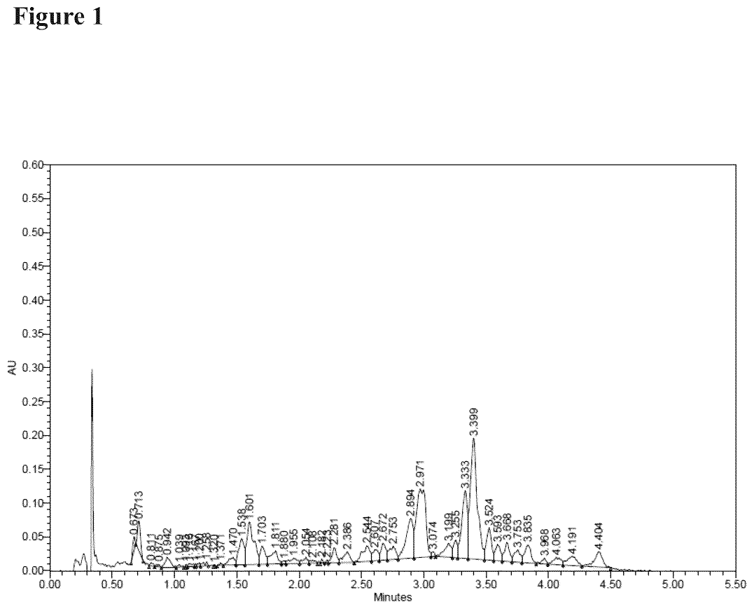

A spray-dried and highly purified extract of saponins of Quillaja (VaxSap®) was employed as starting material for the preparation of an extract containing Compounds I and II from Quillaja saponaria Molina, FIG. 1 shows a representative RP-UHPLC chromatogram of VaxSap.

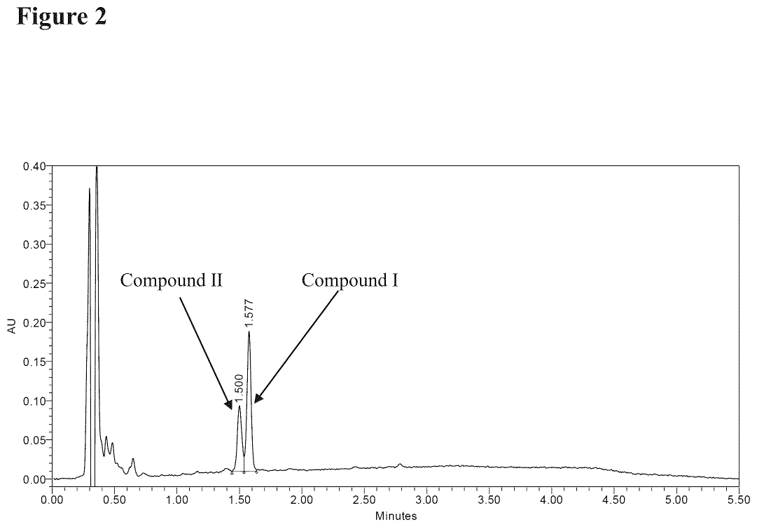

Twenty (20) grams of VaxSap® were suspended in 1000 ml of KOH 28 g/L in aqueous ethanol (50% v/v) and heated at reflux temperature for 5 hours. Once completed the reflux, the pH of the resultant solution was adjusted to the range 6-7 with hydrochloric acid 6M rendering the crude saponin hydrolysate. After drying of 1 L of crude hydrolysate at 50° C. in a heating oven, 48.2 g dried crude saponin hydrolysate were recovered. The conversion of Quillaja saponaria saponins to a mixture containing Compounds I and II was verified by RP-UHPLC chromatography, as shown in FIG. 2.

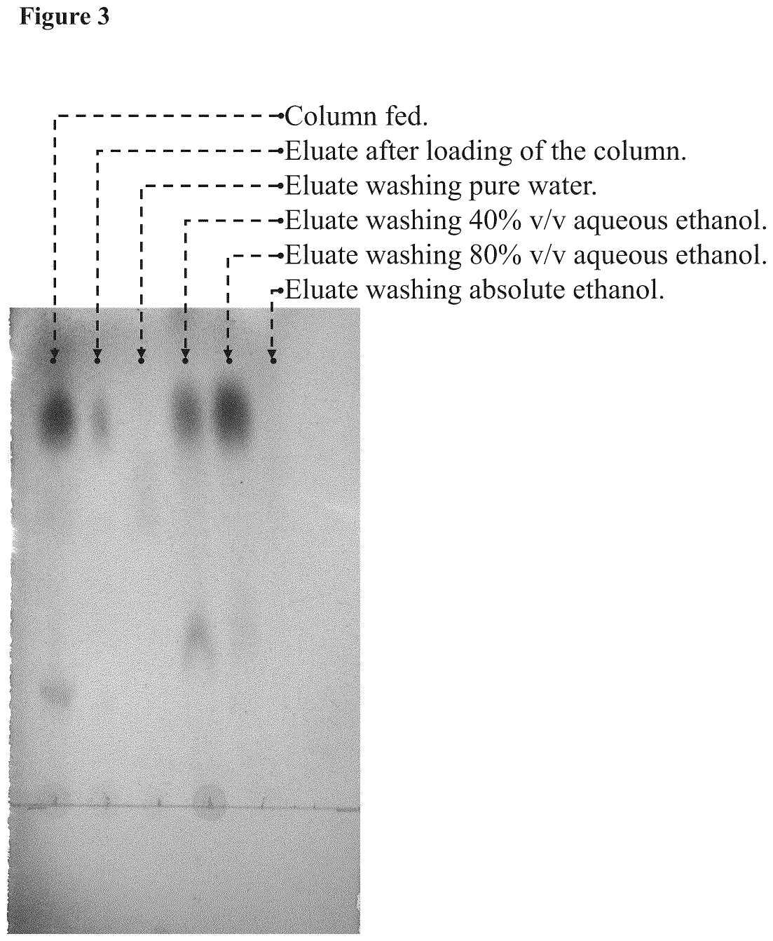

The dried crude saponin hydrolysate was fractionated by column chromatography on a hydrophobic vinyl-divinyl benzene resin. In a representative example, 20 g of hydrophobic vinyl-divinyl benzene resin (Diaion HP-20 resin), were soaked in approximately 2.5 volumes of absolute ethanol for 24 hours. The resultant slurry was transferred to a glass column supplied with a stopcock valve (diameter 1.9 cm; length 30 cm). The column bed (approximately 51 ml) was washed with 250 ml of deionized water. The dried crude saponin hydrolysate (6.4 g) was dissolved in 64 ml of deionized water and acidified to pH 3 by addition of 1.28 ml of formic acid. The resultant solution was fed to the column at an approximate flow rate of 5 ml/min. The loaded crude saponin hydrolysate was fractionated by serial elution with 150 ml of water, 150 ml of aqueous ethanol 40% v/v, 150 ml aqueous ethanol 80% v/v and 150 ml of absolute ethanol. The relative content of Compounds I and II in each eluate was verified by TLC chromatography following the procedure described in Example 3. According to the results of the TLC test, the eluate fraction having the highest content of Compounds I and II was the eluate collected after elution with aqueous ethanol 80% v/v as shown in FIG. 3. The eluate corresponding to the washing with aqueous ethanol 80% v/v was dried in rotary evaporator (70° C.), rendering a syrup residue. By addition of chloroform, the residual water in the syrup residue was removed by evaporation, rendering 0.73 g of hydrolyzed extract of saponins containing the Compounds I and II. The total mass yield of the process described was 0.275 grams of the extract containing Compounds I and II per each gram of VaxSap®.

### Example 5: Preparation of Solid and Liquid Media for Cultivation of Microorganisms

The liquid and solid media employed for the propagation of microorganisms and biotransformation of triterpenic substrates described in the upcoming examples presented in this disclosure is described as follows:

Liquid media: All liquid media employed were sterilized by autoclaving at 121° C. during 20 min prior to use. When the addition of antibiotics to the media was required, they were prepared in separated stock solutions, filtered through sterile syringe filters (pore size 0.22 micron) and added to the autoclaved broth after cooling.
Sterile base broth (SBB) was prepared in deionized water (1 L) containing 10.0 g tryptone, 10.0 g sodium chloride and 5 g yeast extract.
Sabouraud dextrose broth (SDB) was prepared with deionized water (1 L) and contained: 40.0 g glucose, 5.0 g casein and 5.0 g peptone.
Brain Heart Infusion broth (BHIB) was prepared with deionized water (1 L) and contained: 12.5 g brain infusion solids, 5.0 g beef heart infusion solids, 10.0 g proteose peptone, 5.0 g sodium chloride, 2.0 g glucose and 2.5 g disodium phosphate.

Solid media: All media employed were sterilized by autoclaving at 121° C. during 20 min prior to distribution in sterile Petri dishes. When the addition of antibiotics to the media was required, they were prepared in separated stock solutions, filtered through sterile syringe filters (pore size 0.22 micron) and added to the autoclaved broth after partial cooling just before to pouring the agar plates.
Luria Bertani Agar (LBA) was prepared with deionized water (1 L) and contained: 10.0 g tryptone, 5.0 g yeast extract, 10.0 g NaCl and 20.0 g agar-agar.
Trypticase Soy Agar (TSA) was prepared with deionized water (1 L) and contained: 15.0 g pancreatic digest of casein, 5.0 g peptic digest of soybean meal, 5.0 g sodium chloride and 15.0 g agar-agar.
Müller Hinton Agar (MHA) was prepared with deionized water (1 L) and contained: 2.0 g beef extract, 17.5 g acid hydrolysate of casein, 1.5 g starch and 17.0 g agar-agar.
Columbia Agar (CBIA) was prepared with deionized water (1 L) and contained: 10.0 g peptone from casein, 5.0 g peptone from meat, 3.0 g heart extract, 5.0 g extract from yeast, 1.0 g starch, 5.0, sodium chloride and 13.0 g agar-agar.
De Man, Rogosa and Sharpe Agar (MRSA) was prepared with deionized water (1 L) and contained: 10.0 g enzymatic digest of casein, 10.0 g meat extract, 4.0 g yeast extract, 2.0 g tri-ammonium citrate, 5.0 g sodium acetate, 0.2 g magnesium sulfate heptahydrate, 0.05 g manganese sulphate tetrahydrate, 2.0 g di-potassium hydrogen phosphate, 1.08 g sorbitan mono-oleate, 20.0 g glucose and 12.4 g agar-agar.
Sabouraud Dextrose Agar (SDA) was prepared with deionized water (1 L) and contained: 40 g glucose, 5.0 g casein, 5.0 g peptone and 15 g agar-agar.
Brain Heart Infusion Agar (BHIA) was prepared with deionized water (1 L) and contained: 12.5 g brain infusion solids, 5.0 g beef heart infusion solids, 10.0 g proteose peptone, 5.0 g sodium chloride, 2.0 g glucose, 2.5 g disodium phosphate and 10.0 g agar-agar.
Potato Dextrose Agar (PDA) was prepared with deionized water (1 L) and contained: 200 g potato infusion, 20.0 g dextrose and 20 g agar-agar.

### Example 6: Isolation and Propagation of HNR2 Consortium

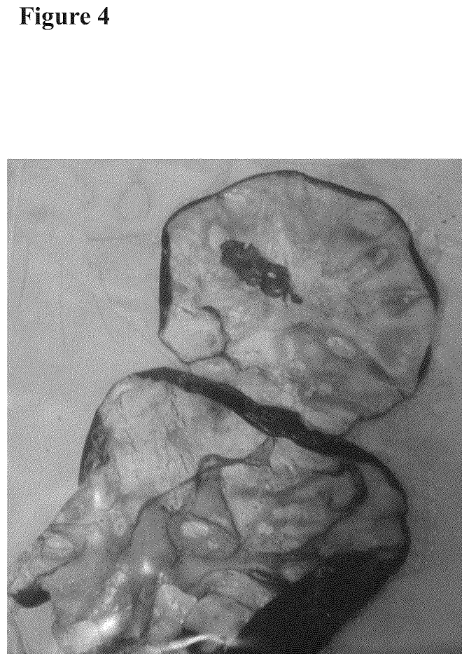

An unpasteurized sample of purified aqueous extract of Quillaja saponaria Molina was employed as the source for the isolation of the consortium HNR2. This aqueous extract of Quillaja saponaria Molina, stored at room temperature, became spoiled by microbial growth evidenced by a gel like substance floating at the surface of the liquid in one pail of the aqueous extract of Quillaja saponaria Molina (FIG. 4). The gel like substance floating at the surface of the aqueous extract of Quillaja saponaria Molina was cut into small pieces with a sterile knife cutter (app. 1×1 cm) on a sterile surface and then transferred to Erlenmeyer flasks containing sterile base broth (prepared as described in the Example 5). The flasks containing the inoculated basic broth were incubated overnight at 25° C. on a rotary shaker at 200 rpm. The liquid culture thus obtained (HNR2 consortium) was kept refrigerated at 4° C. and regrown in fresh basic broth under the same conditions described above on weekly basis.

### Example 7: Characterization of the Consortium HNR2

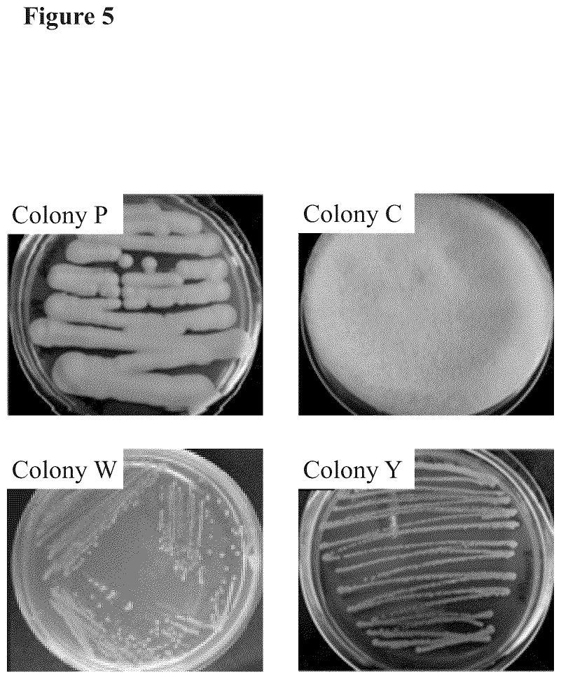

To characterize the HNR2 microbial consortium, 200 microliters aliquots of seed inoculum prepared according to the procedure described in the Example 6 were plated onto Petri dishes containing Luria Bertani Agar (LBA) (prepared as described in the Example 5). The inoculated solid media in Petri dishes were incubated between at 25° C. during 24 to 48 h. Four major types of colonies were grown under the above incubation conditions, hereinafter called colonies C, P, W and Y (FIG. 5).

Growth on solid media: In order to test the growth of colonies C (RGM 2984), P (RGM 3031), W (RGM 3032) and Y (RGM 3021) in different solid media, they were plated on the following solid media described in the Example 5: Trypticase soy agar (TSA), Muller-Hinton agar (MHA), Columbia agar (CBIA), De Man, Rogosa and Sharpe agar (MRSA), Sabouraud dextrose agar (SDA), brain heart infusion agar (BHIA), and potato dextrose agar (PDA). The inoculated solid media in Petri dishes were incubated between at 25° C. during 24 to 48 h. The growth behavior of these colonies is summarized in the Tables 2 and 3.

TABLE 2
Growth of colonies C, P, W and Y in solid media after 24
hours at 25° C. The growth was qualitatively classified
as significant (++), slight (+) and non-observable (−).

| Colony | LBA | TSA | MHA | CBIA | MRSA | SDA | BHIA | PDA |
|---|---|---|---|---|---|---|---|---|
| W | + | ++ | + | + | − | − | + | − |
| Y | + | + | + | + | − | − | + | − |
| P | + | + | + | + | − | − | + | + |
| C | ++ | ++ | ++ | ++ | ++ | ++ | ++ | ++ |

TABLE 3
Growth of colonies C, P, W and Y in solid media after 48
hours at 25° C. The growth was qualitatively classified
as significant (++), slight (+) and non-observable (−).

| Colony | LBA | TSA | MHA | CBIA | MRSA | SDA | BHIA | PDA |
|---|---|---|---|---|---|---|---|---|
| W | + | ++ | + | ++ | − | − | ++ | − |
| Y | ++ | ++ | + | ++ | − | − | ++ | − |
| P | + | + | ++ | ++ | ++ | + | + | ++ |
| C | ++ | ++ | ++ | ++ | ++ | ++ | ++ | ++ |

Growth on solid media supplemented with antibiotics: In order to test the resistance to chloramphenicol, ampicillin, kanamycin and streptomycin of the colonies C, P Wand Y during the growth in solid media, they were plated onto Petri dishes containing Luria Bertani Agar (colonies W and Y) and potato dextrose agar (colonies C and P) supplemented with antibiotics as described in the Tables 4 and 5, and incubated 48 hours at 25° C.

TABLE 4
Growth of colonies C and P on solid media after 48 hours at 25° C.

| Solid media | Colony C | Colony P |
|---|---|---|
| PDA + chloramphenicol 20 microg/ml | Resistant | Resistant |
| PDA + ampicillin 20 microg/ml | Resistant | Resistant |
| PDA + kanamycin 20 microg/ml | Resistant | Resistant |
| PDA + streptomycin 20 microg/ml | Resistant | Resistant |

TABLE 5
Growth of colonies W and Y on solid
media after 48 hours at 25° C.

| Solid media | Colony W | Colony Y |
|---|---|---|
| LBA + chloramphenicol 20 microg/ml | Sensitive | Sensitive |
| LBA + ampicillin 20 microg/ml | Resistant | Resistant |
| LBA + kanamycin 20 microg/ml | Sensitive | Resistant |
| LBA + streptomycin 20 microg/ml | Resistant | Sensitive |

Morphology and biochemical characterization: The morphology of the colonies C, P W and Y was determined after growth on Petri dishes containing Luria Bertani Agar (colonies W and Y) and potato dextrose agar (colonies C and P). Samples of each type of colony were used for morphology and biochemical studies following the procedures in Bergey's Manual® of Systematic Bacteriology. The results are summarized in the Table 6.

TABLE 6
Summary of the characterization of the characterization
of the colonies C, P (grown on PDA medium), and W,
Y (grown on LDA medium) after 48 hours at 25° C.

| Parameter | Colony C | Colony P | Colony W | Colony Y |
|---|---|---|---|---|
| Chromogenesis | White | Pink | White | Yellow |
| Shape of the | Rhizoid | Circular | Filamentous | Circular |
| colony | | | | |
| Surface of the | Umbonate | Convex | Flat | Convex |
| colony | | | | |
| Edge of the | Filiform | Entire | Undulate | Entire |
| colony | | | | |
| Opacity | Opaque | Opaque | Translucent | Opaque |
| Gram staining/ | Not | Not | Gram | Gram |
| shape observed | determined | determined | Negative/ | Negative/ |
| in the | | | bacillus | coccus |
| microscope | | | | |
| Lactophenol | Positive | Positive | Not | Not |
| cotton blue | staining/ | staining/ | determined | determined |
| staining/shape | filamentous | spherical | | |
| observed in the | | | | |
| microscope | | | | |
| Catalase | Not | Not | Negative | Positive |
| | determined | determined | | |
| Amylase | Not | Not | Positive | Negative |
| | determined | determined | | |
| Gelatinase | Not | Not | Negative | Negative |
| | determined | determined | | |

Characterization by ribosomal sequence analysis: In order to complete the characterization of the colonies C, P, W and Y the ribosomal sequence analysis was performed as follows:

Microbial propagation: A single colony of C, P W and Y strains was inoculated in 4 ml of the media listed in the Table 7, and grown overnight at 25° C. on a rotary shaker at 200 rpm.

TABLE 7
Liquid culture media employed for the propagation of the colonies C, P, W and
Y as required for their characterization by ribosomal sequence analysis.

| | | | | Chloram- | | |
|---|---|---|---|---|---|---|
| | | Glucose | Ampicilin | phenicol | Kanamycin | Streptomycin |
| | | (added as | (added as | (added as | (added as | (added as |
| | | sterile stock | sterile stock | sterile stock | sterile stock | sterile stock |
| | | solution to | solution to | solution to | solution to | solution to |
| | | the autoclaved | the autoclaved | the autoclaved | the autoclaved | the autoclaved |
| Strain | Broth | broth) | broth) | broth) | broth) | broth) |
| W | SBB | 10 g/L | — | — | — | 20 mg/L |
| Y | SBB | 10 g/L | — | — | 20 mg/L | — |
| P | SDB | 10 g/L | — | 20 mg/L | — | — |
| C | SBB | 10 g/L | 50 mg/L | 20 mg/L | — | — |

Total DNA isolation: Each culture was centrifuged 5 min at 20,000×g. Total genomic PG 37T DNA was isolated from the centrifuged pellets using two commercial kits: E.Z.N.A.® Bacterial DNA Kit (Omega Bio-Tek) for colonies W and Y, and DNeasy Plant Mini Kit (QIAGEN) for colonies C and P. In each case, the DNA was isolated following the instruction of the manufacturer. The DNA of each colony was recovered in 100 μL of buffer TE (Tris 10 mM, EDTA 1 mM, pH 8.0). The purity and yield of the DNA preparations were determined by measurement of the absorbance at 260 and 280 nm of each DNA solution. The DNA solutions were stored at −20° C.

Primers for amplification of ribosomal sequences from total DNA by polymerase chain reaction (PCR): Table 8 summarizes the nucleotide sequences of the DNA primers employed for the amplification of ribosomal sequences from total DNA isolated from colonies C, P, W and Y.

TABLE 8
Nucleotide sequences of the DNA primers
employed for the PCR amplification of
ribosomal sequences from total DNA.

| | | | | Expected |
|---|---|---|---|---|
| | | | | amplicon |
| | | Nucleotide | | size |
| | Primer | sequence | Target | [base |
| Pair. | name. | (5′→3′). | sequence | pairs] |
| PP1 | 27F | AGAGTTTGATCMTGG | Bacterial | 1450 |
| | | CTCAG | ribosomal | |
| | | (SEQ ID No.: 1) | sequence | |
| | R1492 | TACGGYTACCTTGTT | 16S | |
| | | ACGACTT | rDNA (*) | |
| | | (SEQ ID No.: 2) | | |
| PP2 | 63F | CAGGCCTAACACATG | Bacterial | 1500 |
| | | CAAGTC | ribosomal | |
| | | (SEQ ID No.: 3) | sequence | |
| | M1387R | GGGCGGWGTGTACAA | 16S | |
| | | GRC | rDNA (*) | |
| | | (SEQ ID No.: 4) | | |
| PP3 | NL-1 | GCATATCAATAAGCG | Fungal | 680 |
| | | GAGGAAAAG | D1/D2 | |
| | | (SEQ ID No.: 5) | region of | |
| | NL-4 | GGTCCGTGTTTCAAG | the | |
| | | ACGG | ribosomal | |
| | | (SEQ ID No.: 6) | sequence | |
| | | | 28S | |
| | | | rDNA (**) | |
| PP4 | ITS1 | TCCGTAGGTGAACCT | Fungal | 620 |
| | | GCGG | ribosomal | |
| | | (SEQ ID No.: 7) | sequence | |
| | ITS4 | TCCTCCGCTTATTGAT | 5S rDNA | |
| | | ATGC | (***) | |
| | | (SEQ ID No.: 8) | | |

(*) Fredriksson, N. J., Hermansson, M., & Wilén, B. M. 2013. “The choice of PCR primers has great impact on assessments of bacterial community diversity and dynamics in a wastewater treatment plant”. PLoS One. 8(10): e76431.
(**) Fernández-Bodega, M. A., Mauriz, E., Gómez, A., & Martin, J. F. 2009. “Proteolytic activity, mycotoxins and andrastin A in Penicillium roqueforti strains isolated from Cabrales, Valdeón and Bejes-Tresviso local varieties of blue-veined cheeses”. International journal of food microbiology. 136(1): 18-25.
(***) Gardes M, Bruns TD. 1993. “ITS primers with enhanced specificity for basidiomycetes - application to the identification of mycorrhizae and rusts”. Molecular Ecology. 2: 113-118.

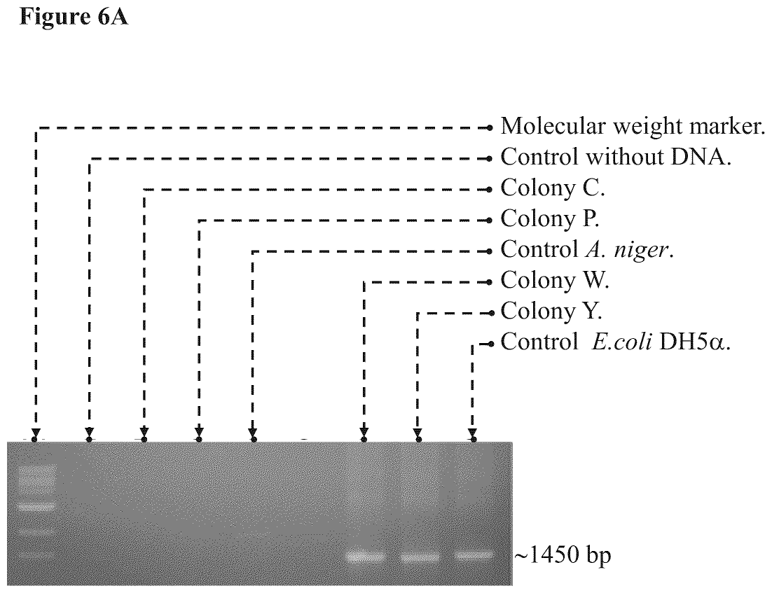

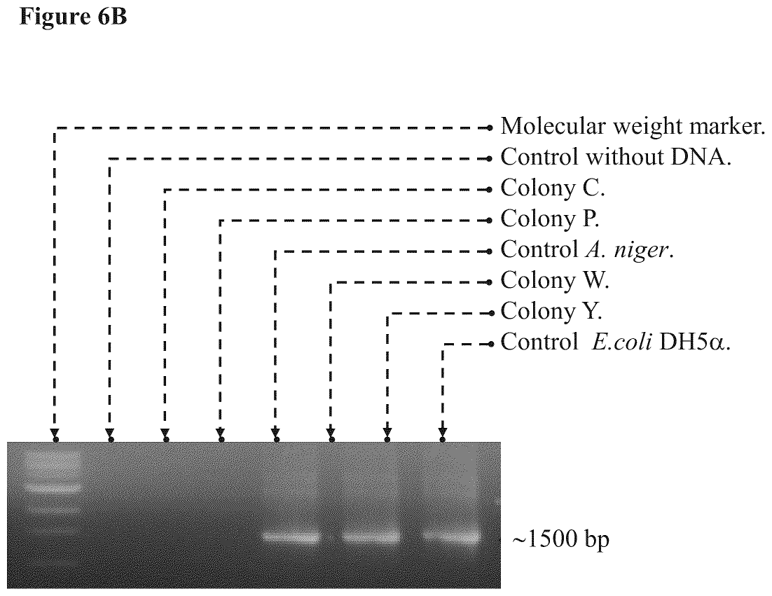

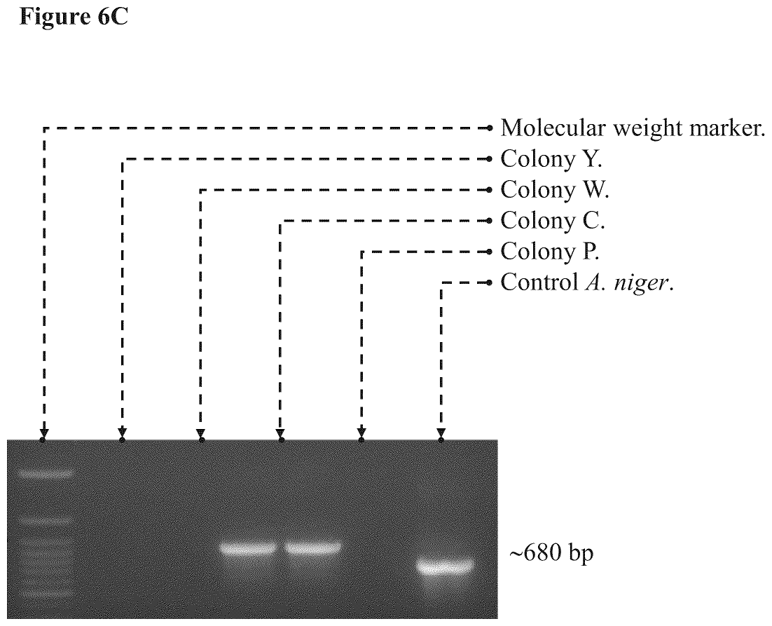

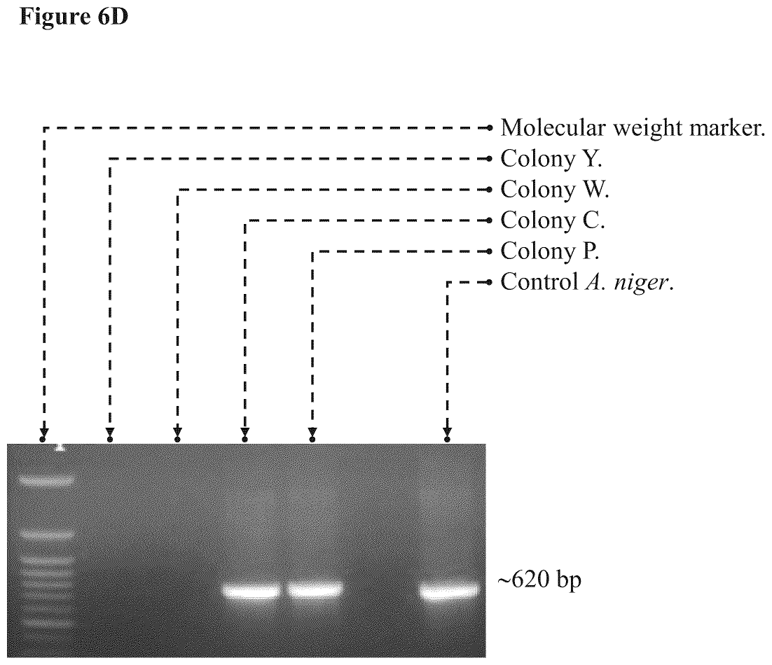

FIGS. 6A, 6B, 6C and 6D show the results of the agarose electrophoresis analysis of the PCR products obtained by amplification of the genomic DNA of the colonies C, P, W and Y, with the primer pairs PP1 (SEQ ID Nos.: 1 and 2), PP2 (SEQ ID Nos.: 3 and 4), PP3 (SEQ ID Nos.: 5 and 6) and PP4 (SEQ ID Nos.: 7 and 8), respectively. The results shown in FIGS. 6A and 6B confirm that colonies W and Y, are prokaryotic bacterial cells. The results shown in FIGS. 6C and 6D confirm that colonies C and P, are fungal eukaryotic cells.

The PCR products described previously were sequenced (Macrogen, Seoul, Korea) (Sequences SEQ ID No. 9 a 23. DNA sequences of the PCR product obtained from genomic DNA of colony C with primer pair PP3 (SEQ ID Nos.: 5 and 6). DNA sequences of the PCR product obtained from genomic DNA of colony C with primer pair PP4 (SEQ ID Nos.: 7 and 8). DNA sequences of the PCR product obtained from genomic DNA of colony P with primer pair PP3 (SEQ ID Nos.: 5 and 6). DNA sequences of the PCR is product obtained from genomic DNA of colony P with primer pair PP4 (SEQ ID Nos.: 7 and 8). DNA sequences of the PCR product obtained from genomic DNA of colony W with primer pair PP1 (SEQ ID Nos.: 1 and 2). DNA sequences of the PCR product obtained from genomic DNA of colony W with primer pair PP2 (SEQ ID Nos.: 3 and 4). DNA sequences of the PCR product obtained from genomic DNA of colony Y with primer pair PP1 (SEQ ID Nos.: 1 and 2). DNA sequences of the PCR product obtained from genomic DNA of colony Y with primer pair PP2. (SEQ ID Nos.: 3 y and 4)). The DNA sequences were edited and aligned against published ribosomal sequences of prokaryotic and eukaryotic microorganisms employing the software NCBI BLAST (http://www.ncbinlm.gov/) and ClustalW (multiple sequence alignment in BioEdit v7.0.9 (http://www.mbio.nesu.edu/BioEdit/bioedit.html). The list of the most probable microorganisms corresponding to each type colony under study is summarized in the Table 9. According to the results, colonies C, P, W and Y most likely correspond to Mucor spp, Rhodotorula spp, Paenibacillus spp and Microbacterium spp, respectively.

TABLE 9
List of the most probable microorganisms corresponding
to the colonies C, P, W and Y, as determined by alignment
of ribosomal sequences employing CLUSTAL W.

| | | Sequence homology (%) | | | |
|---|---|---|---|---|---|
| | | Primer | Primer | Primer | Primer |
| | | pair PP1 | pair PP2 | pair PP3 | pair PP4 |
| | Probable type of | (SEQ ID | (SEQ ID | (SEQ ID | (SEQ ID |
| | microorganism according to | Nos.: 1 | Nos.: 3 | Nos.: 5 | Nos.: 7 |
| Colony | sequence alignment | and 2) | and 4) | and 6) | and 8) |
| C | Mucor racemosus strain | — | — | 100% | 99% |
| | ATCC 42647 | | | | |
| C | Mucor racemosus strain | — | — | 100% | 99% |
| | X1650 | | | | |
| P | Rhodotorula mucilaginosa | — | — | 95% | 100% |
| P | Rhodotorula mucilaginosa | — | — | 95% | 99% |
| W | Paenibacillus amylolyticus | 99% | 80% | — | — |
| W | Paenibacillus tylopili | 99% | 80% | — | — |
| W | Paenibacillus tundrae | 99% | 80% | — | — |
| Y | Microbacterium | 99% | 89% | — | — |
| | phyllosphaerae | | | | |
| Y | Microbacterium | 99% | 84% | — | — |
| | hidrocarbonoxydans | | | | |
| Y | Microbacterium foliomm | 99% | 84% | — | — |

### Example 8: Production of SPH03 from an Extract Comprising Compounds I and II

To produce the seed inoculum, an aliquot of a refrigerated culture HNR2 as defined above, was inoculated on 5 ml of sterile base broth prepared according to the recipe described in the Example 5. The cells were grown overnight at 25° C. on a rotary shaker at 200 rpm, pelleted by centrifugation and washed with 5 ml of sterile base broth. Washed cells were resuspended on 5 ml of sterile base broth rendering the seed inoculum.

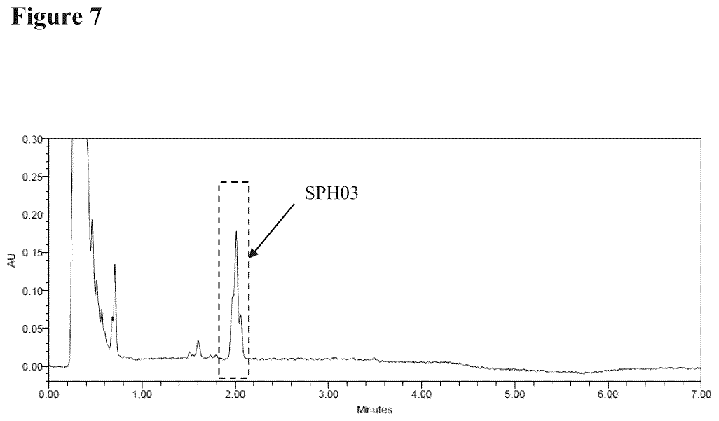

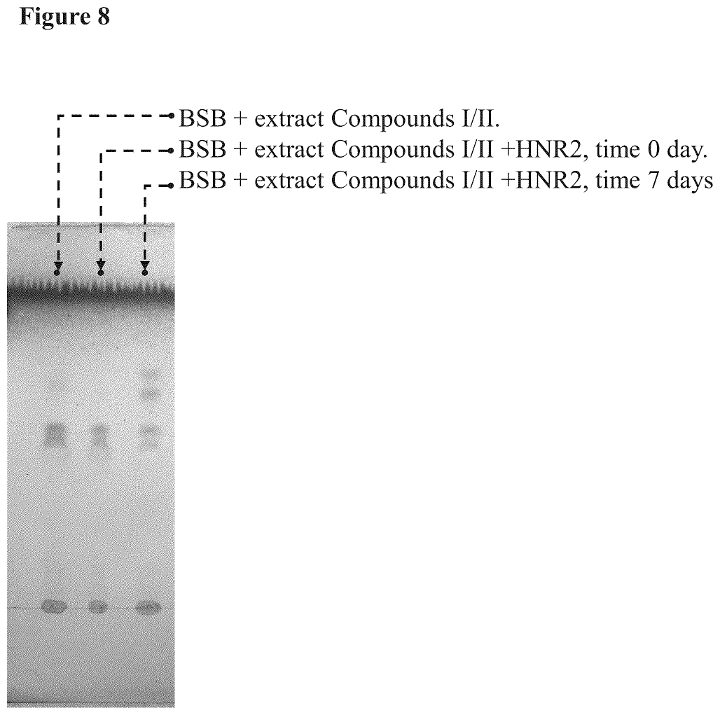

To perform the biotransformation of the hydrolyzed extract of saponins of Quillaja saponaria Molina, shake flasks (250 ml) containing 45 ml of base broth were sterilized at 1210° C. for 20 min. After sterilization, 100 microliters of filter sterilized glucose (200 g/L) and 2.0 ml of filter sterilized an extract comprising Compounds I and II (24 g/L) were blended into the sterilized base broth. The flasks were cooled to 25° C. and inoculated with 1.5 ml of seed inoculum prepared according to the procedure described in the Example 6. The flasks were then incubated at 25° C. on a rotary shaker (200 rpm) during one week and sampled at various intervals. One (1) ml samples were removed and centrifuged to remove cells. Progress of the biotransformation was followed by UHPLC-PDA and TLC analysis of the supernatants of each sample, according to the procedure described in the Examples 1 and 3. The UHPLC analysis of the supernatant of the culture shows no trace of Compounds I and II, and the rising of a new peak with increased retention time, hereinafter denominated as SPH03 is shown in FIG. 7, confirms the biotransformation of the extract containing the Compounds I and II by HNR2 culture. Similarly, TLC analysis of culture supernatants, also showed the disappearance of the Compounds I and II, and the appearance of SPH03 as shown in FIG. 8.

### Example 9: Effect of Broth Composition on the Production of SPH03 from an Extract Containing Compounds I and II

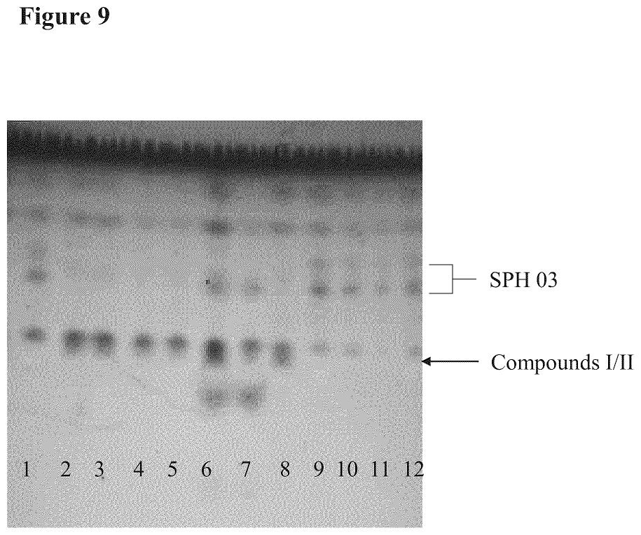

The impact of the broth formulation on SPH03 production from partially hydrolyzed saponins (Compounds I and II) during the biotransformation process was assessed on a range of liquid broths based on SBB and BHIB broths (Example 5) supplemented with some carbohydrates as described in the Table 10. SPH03 production in the cultures is formulated according to the Table 10 was determined by TLC chromatography (FIG. 9).

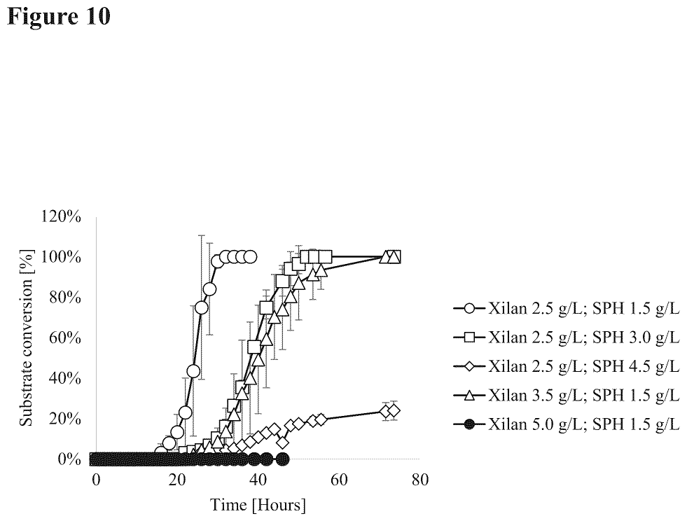

TABLE 10
Effect of the broth formulation on the production of SPH03
from an extract containing Compounds I and II, catalyzed
by the HNR2 consortium: Results after 120 h of incubation.

| | | | Extract | | Time for |
|---|---|---|---|---|---|
| TLC | | | containing | | complete |
| lane | Liquid broth | Inoculum | Compounds | Production | conversion |
| FIG. 10 | composition | HNR2 | I and II | of SPH03? | [h] |
| 1 | SBB | + | 1 g/L | + | 48 |
| 2 | SBB + glucose 1.0 g/L | + | 1 g/L | − | − |
| 3 | SBB + sucrose 1.0 g/L | + | 1 g/L | − | − |
| 4 | SBB + lactose 1.0 g/L | + | 1 g/L | − | − |
| 5 | SBB | − | 1 g/L | − | − |
| 6 | BHIB | + | 1 g/L | − | − |
| 7 | BHIB + glucose 0.4 g/L | + | 1 g/L | − | − |
| 8 | SBB + sucrose 0.4 g/L | + | 1 g/L | − | − |
| 9, 10 | SBB + xylan 1.0 g/L | + | 1 g/L | + | 36 |
| 11 | SBB + lactose 0.4 g/L | + | 1 g/L | + | 96 |
| 12 | SBB + glucose 0.4 g/L | + | 1 g/L | + | 48 |

According to the results shown in Table 10, BHIB broth as well as the addition of sucrose 0.4 g/L to SBB inhibits the production of SPH03. The addition of glucose and lactose 1.0 g/L inhibits SPH3 production. The quickest conversion of the Compounds I and II to SPH03 is achieved in SBB broth supplemented with xylan 1 g/L. The impact of the substrate concentration for the biotransformation (extract containing Compounds I and II) and xylan on SPH3 production during the biotransformation process, was assessed on a range of SBB based broths formulated as described in the Table 11. The conversion of extract containing Compounds I and II in SPH03 in the cultures formulated according to the Table 11 was determined by UHPLC chromatography of samples taken at regular time intervals (FIG. 10).

TABLE 11
Effect xylan and Compounds I and II concentrations
on the production of SPH03 from an extract containing
Compounds I and II, catalyzed by HNR2 consortium.

| | | | | Productivity |
|---|---|---|---|---|
| Extract | | | | of SPH03 at |
| containing | | Conversion to | Time for | the time for |
| Compounds I | | SPH03 | maximum | maximum |
| and II | Xylan | [conversion | conversion | conversion |
| [g/L] | [g/L] | %] | [h] | [mg/L/h] |
| 1.5 g/L | 2.5 g/L | 100% | 32 | 47 |
| 3.0 g/L | 2.5 g/L | 100% | 52 | 58 |
| 4.5 g/L | 2.5 g/L | 24% | 73 | 15 |
| 1.5 g/L | 3.5 g/L | 100% | 71 | 21 |
| 1.5 g/L | 5.0 g/L | 0% | ND | 0 |

According to the results shown in Table 11, SBB broth formulated with 3.0 g/L of extract containing Compounds I and II, and 2.5 g/L xylan allows to maximize the SPH03 production in batch cultures of HNR2 consortium.

### Example 10: Purification of SPH03 from the Biotransformation Broth

As a representative example of SPH03 recovery we provide a detailed description of the purification procedure as applied to the recovery of the biotransformation product from the cultivation broth of HNR2 grown on SBB broth formulated with 1.0 g/L of extract containing Compounds I and II, and 2.5 g/L xylan. The following description is not limited to the purification of SPH03 from the cultivation broth obtained in the present examples as well as the Examples 8 and 9, but also applicable to the processing of biotransformation broths obtained in other conditions. The cultivation broth (300 ml) was centrifuged at 3220×g for 10 min in order to remove the microbial cells. The supernatant is was recovered, and the pH adjusted to 5.5 by addition of formic acid 1 M and 5% v/v of acetonitrile. The resulting solution was concentrated to ⅕ of the initial volume by evaporation at reduced pressure at 36° C. rendering a concentrated precursor of SPH03. Aliquots of the concentrated precursor of SPH03 (1.4 ml) were injected in a semipreparative reverse phase HPLC column (inner diameter 21.2 mm; length 250 mm; particle size 7 μm) and eluted in gradient mode at constant flow rate (30 ml/min) employing the following mobile phases: Solvent A: Formic acid 0.15% v/v in water, and; Solvent B: Formic acid 0.15% v/v in acetonitrile. The solvent gradient employed is described as follows: Step 1: 0-0.4 min 5% of Solvent B; Step 2: 0.4-24 min from 5% to 40% of Solvent B; Step 3: 24-35 min 40% of Solvent B; Step 4, and; 35-47 min from 40% to 5% of Solvent B.

The saponin profile of the collected fractions is then determined by reverse phase UHPLC according to the conditions described in the Example 1. Fractions containing the target saponin were selected for further purification. The selected fractions of 45 preparative runs were pooled and solvent was removed by evaporation at reduced pressure at 35° C. The residual aqueous solution was lyophilized, rendering the purified SPH03.

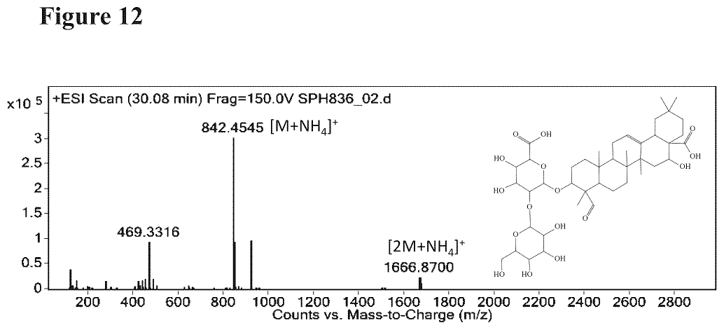

At the end of the drying procedure 88 mg of purified SPH03 were recovered. The total mass yield of the process described was 0.29 g of SPH03 per g of an extract containing Compounds I and II. FIG. 12 provides a representative RP UHPLC chromatogram of the SPH03 product purified by preparative RP HPLC.

### Example 11: Identification of Triterpenic Components in SPH03

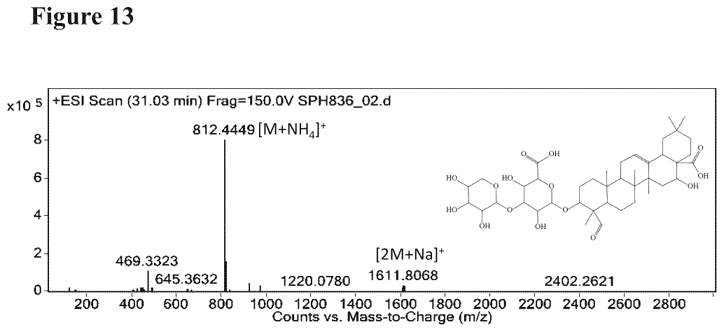

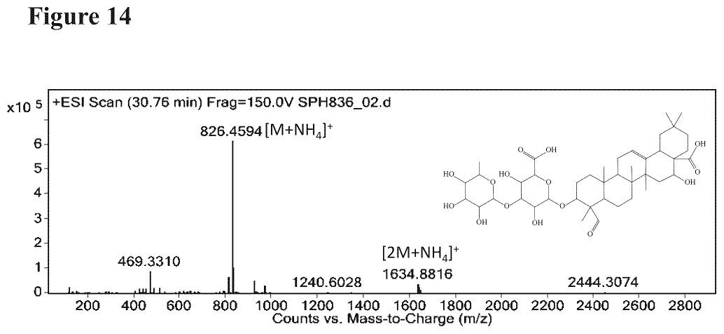

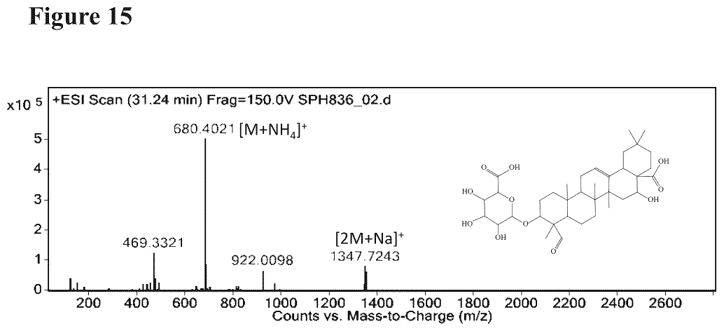

The components in SPH03 were identified by HPLC-ESI-TOF-MS as described in the is Example 2. In the SPH03 fraction, four co-eluting compounds were found, herein referred as Compounds III, IV, V and VI. The mass of the pseudo-molecular ions [M+NH₄]⁺ was detected in the mass spectra of Compounds III, IV, V and VI, as shown in FIGS. 13, 14, 15 and 16, respectively. The molecular mass of the ammonium adducts and acid forms of Compounds HI, IV, V and VI, are summarized in the Table 12.

TABLE 12
Molecular mass of the ammonium adducts and acid forms of
Compounds III, IV, V and VI detected by HPLC-ESI-TOF-MS.

| | Molecular mass of | Molecular mass of |
|---|---|---|
| | the ammonium adducts. | the acid form. |
| Compound | [Dalton] | [Dalton] |
| III | 842 | 825 |
| IV | 812 | 795 |
| V | 826 | 809 |
| VI | 680 | 663 |

Compound IIII has been referred previously in the literature as a minor saponin in the extracts of the bark of Quillaja saponaria Molina, and as degradation product during acid hydrolysis of the saponins of quillaja Guo et al., Phytochemistry. 48: 175-180 (1998). Compound VI has been described by Labriola & Deulofeu (Experientia. 25: 124-125 (1969)) as a product of acid hydrolysis of Quillaja saponins with hot acid. The Compounds IV and V have not been previously detected in quillaja extracts.

### Example 12: Hemolytic Activity of SPH03

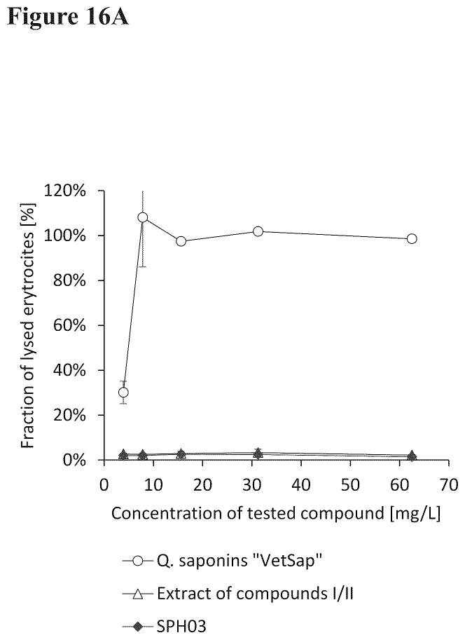

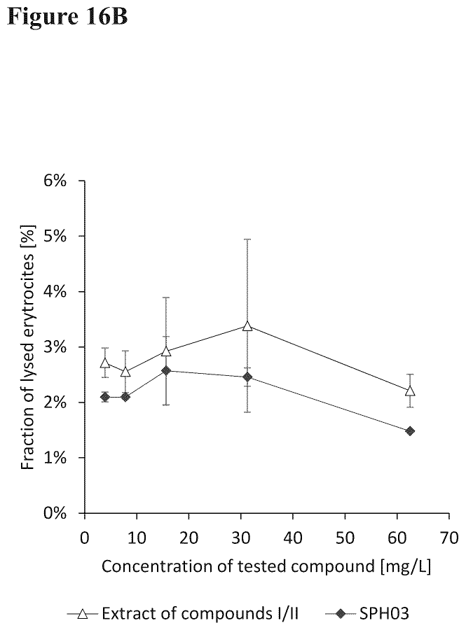

The hemolytic activity of SPH03, an extract containing Compounds I and II and purified Quillaja saponins “VetSap” (Desert King International) was determined by the following method: Serial dilutions of each sample in PBS buffer were made in Eppendorf tubes (250 microL). All samples were tested in duplicate. Chicken red blood cells (CRBC) were washed three times with PBS, and were diluted to 2% with PBS. CRBC (250 microL) were added to each tube. A mixture of Quillaja saponins “VetSap” 500 mg/L (250 microL) and CRBC (250 microL) was prepared as positive control. A mixture of PBS (250 microL) and CRBC (250 microL) was prepared as negative control. The contents of all tubes were gently mixed by inversion and incubated at 37° C. by 1 h. After incubation, the tubes were spun at 18,620×g to sediment non hemolyzed cells. 100 microL of the supernatant from each tube was transferred to separate wells in a flat bottom microtiter plate. Absorbance was determined at 540 nm against water blank with an Epoch microplate spectrophotometer. (BioTek Instruments Inc.). Hemolysis increased the absorbance at 540 nm due to release of hemoglobin from the lysed cells. The value of the absorbance of the negative control was subtracted to all remaining absorbance values corresponding to samples and positive control, rendering the ΔAbs values. The hemolysis percentage was determined dividing each ΔAbs by the corresponding ΔAbs of the positive control (FIGS. 16A and 16B). Significant differences in hemolysis were observed between adjuvants the tested samples. Quillaja saponins “VetSap” caused partial hemolysis at concentrations as low as 3.9 mg/L; complete hemolysis was observed with concentrations in the range 7.8-62.5 mg/L (FIG. 16A). Negligible hemolysis (less than 5%) was observed with SPH03 and the extract containing Compounds I and II in the same range of concentrations tested (3.9-62.5 mg/L; FIGS. 16A and 16B).

### Example 13: Stability of SPH03 at Physiological pH

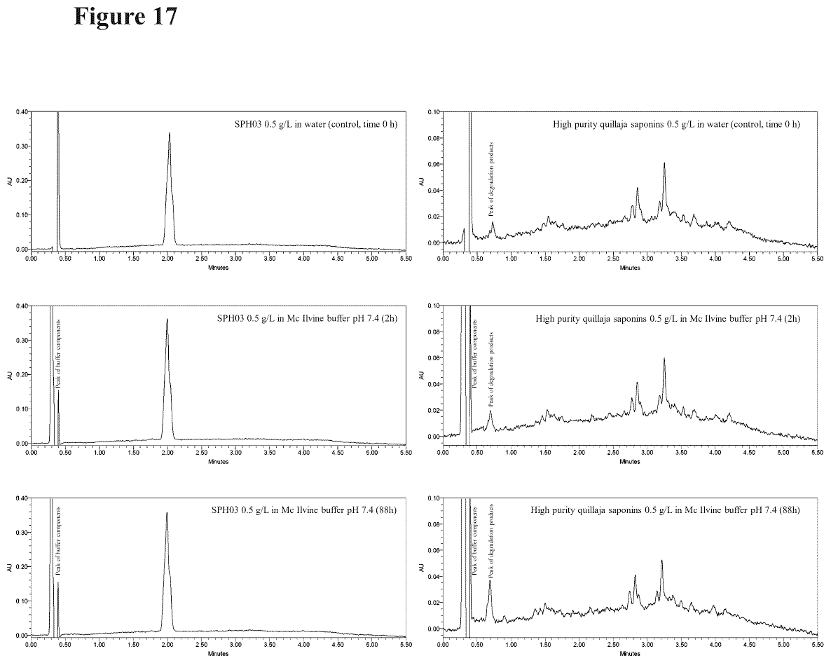

Fresh solutions 0.5 g/L of SPH03 and high purity Quillaja saponins were prepared in Mc Ilvine buffer (pH 7.4) incubated at 37° C. during 88 h. The stability of each solution was determined by UHPLC chromatography of samples taken at 0, 2 and 88 hours. The results confirm the SPH03 stability at physiological pH. Under the same conditions, Quillaja saponins were partially degraded (FIG. 17).

### Example 14: Cytotoxic activity of SPH03

A solution (1 mM) of SPH03 and the extract containing Compounds I and II were prepared on PBS buffer. The cytotoxic activity of SPH03 fraction and the extract containing Compounds I and II were tested on two types of tumor cells of human gastric cancer already available at the American Type Culture Collection (ATCC): SNU1 (gastric carcinoma) and AGS (gastric adenoma), a human gastric epithelial cell line GES-1 used as normal cell control. All the cell lines were grown on RPMI 1640 broth or DEMEN supplemented with 10% FBS and 1% penicillin/streptomycin, under CO₂ rich atmosphere (5%) at 37° C. Grown cells were transferred to fresh broth and propagated until 80% density. Then, the cell culture containing 5,000 cells was transferred to 96 well plates for further growth during 24 h. After growth, the stock solutions of SPH03 and the extract containing Compounds I and II were added to the wells in order to test the cytotoxic effect of each solution in a range of concentrations covering 0 to 150 μM and is 0 to 100 μM, respectively. Cell suspensions were further incubated during 48 and 72 h at 37° C. under CO₂ atmosphere (5%). Cell viability was determined spectrophotometrically at 490 nm using the reagent CellTiter 96@AQueous One Solution Proliferation Assay (MTS) supplied by Promega (Madison, WI, USA). Each condition was tested in triplicate and three independent assays. To obtain IC₅₀ value to SPH03, dose-response curve was constructed to SNU1 and AGS cell lines using GraphPad Prism 8 (GraphPad Software, San Diego, CA, USA).

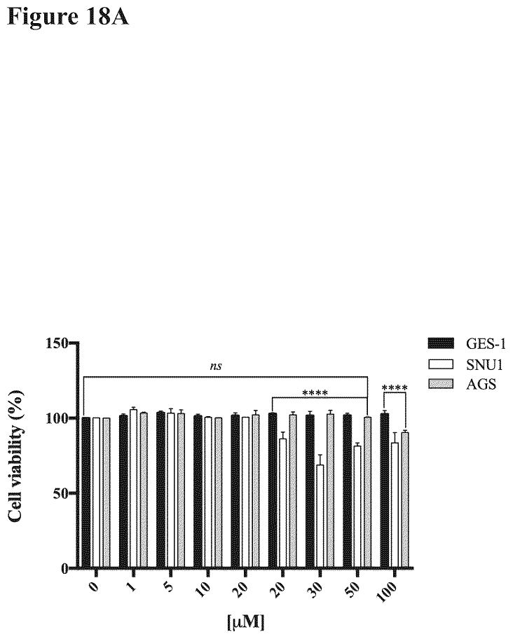

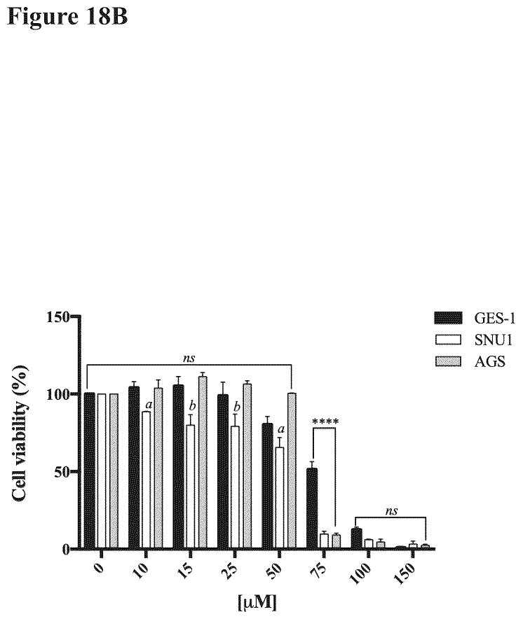

The results of IC₅₀ obtained are shown in Table 13. After incubation during 72 h, the extract containing Compounds I and II had not severe impact on cell viability of AGS and GES-1 cell lines in all the ranges of concentration assayed (FIG. 18a). However, a slight effect was observed on the viability of SNU1 cells in the range 20 to 30 μM. On the other hand, the SPH03 fraction was able to abolish above a 40% of the cell viability of tumoral SNU1 cells at 50 μM (FIG. 18b). When the concentration of SPH03 fraction was increased to 75 μM, the viability of SNU1 and AGS cells was almost abolished (9%), whereas 51% of GES-1 cells remained viable (p<0.0001) (FIG. 18b). The latter results show a significant difference of the cytotoxic effect of SPH03 on normal cell vs cancer cell lines.

TABLE 13
In vitro anti-proliferative activity of an extract containing
Compounds I and II and SPH03 on gastric cancer cell lines (SNU1
and AGS) and normal human gastric cells (GES-1). The IC₅₀
values represent the average of at least 3 independent experiments.

IC₅₀ (μM)

| Tested sample | SNU1 cells | AGS cells | GES-1 cells |
|---|---|---|---|
| SPH03 | 47.72 | 59.94 | 69.09 |
| Extract containing | >100 | >100 | >100 |
| Compounds I and II | | | |

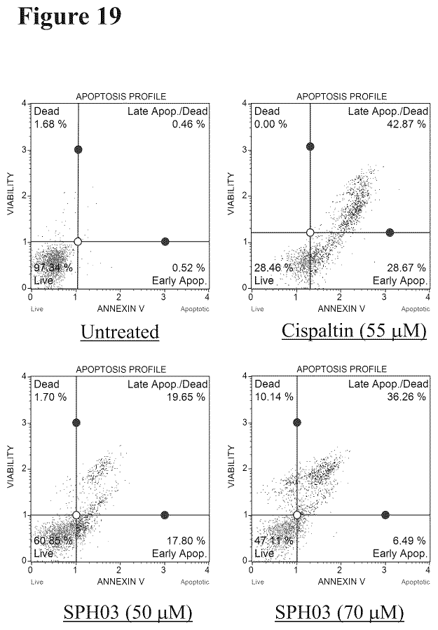

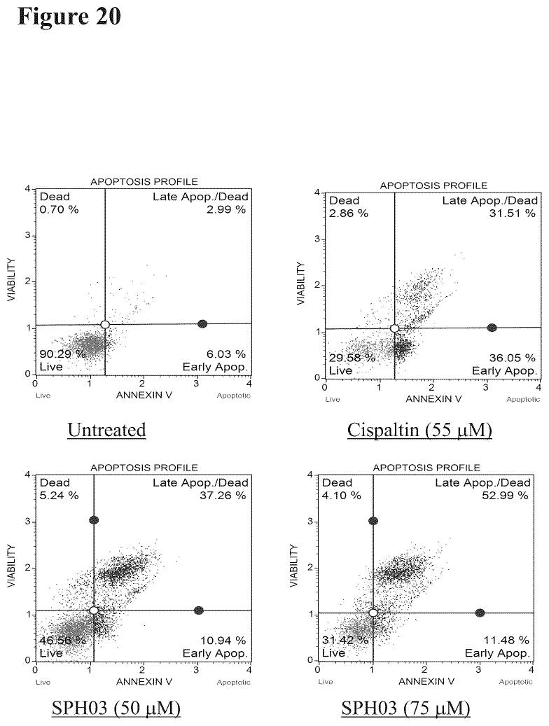

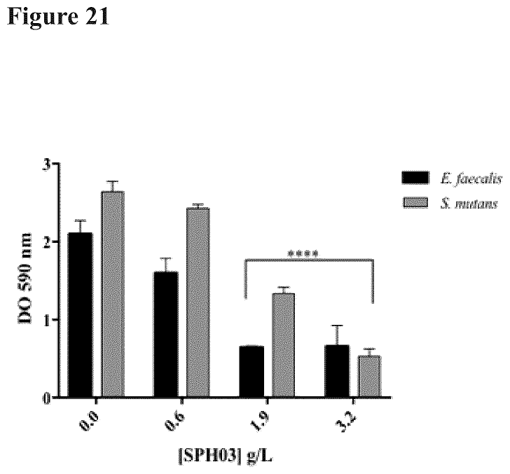

To confirm the pro-apoptotic effect of SPH03, the treated cells were assayed by Muse Annexin V as well as a dead cell kit (Merck, Millipore, USA), according to the procedures recommended by each manufacturer. To this end, 2·10⁵ cells (AGS and SNU1) were seeded in 6-well plates and treated with different concentrations of SPH03 (35, 50 and 75 μM) by 48 h. Cisplatin drug (50 μM) was employed as positive control of apoptosis. Cultured AGS and SNU cells not supplemented with SPH03 were employed as negative control. After incubation, the cells were washed with 1 mL PBS and 100 μL of Muse™ Annexin V and 100 μL of Dead Cell reagent were added. Then, the cells were incubated at room temperature during 20 min in the darkness. The apoptotic effect was measured by using Muse cell analyzer and Muse analysis software (Merck-Merck Millipore), where the cells were classified into four groups: live, early apoptotic, late apoptotic and dead or necrosis. The percentage of AGS cells undergoing apoptotic cell death (early and late stages) increased to 37.45% and 42.8% after exposure to 50 and 70 μM of SPH03, respectively (FIG. 19). Untreated AGS cells cultured as negative control were 97.34% viable (FIG. 19). In the case of SNU1 the percentage of cells undergoing apoptotic cell death (early and late stages) increased to a 40.3 and 64.5%, after exposure to 50 μM and 70 μM of SPH03, respectively (FIG. 20). Untreated SNU1 cells cultured as negative control were 90.29% viable (FIG. 21).

### Example 15: Inhibition of Biofilm Formation by Streptococcus mutans UA159 and Enterecoccus faecalis ATCC 29212 Grown in Broth Supplemented with SPH03

To measure the impact of SPH03 on the formation of biofilms by Streptococcus mutans UA159 and Enterecoccus faecalis ATCC 29212, an assay based on the binding of crystal violet dye to biofilms was applied. Streptococcus mutans UA159 and Enterecoccus faecalis ATCC 29212 were inoculated on BHIB broth (Example 5) and cultured by 18 h at 37° C. in a shaking incubator. Once the optical density at 600 nm (OD600) of each culture reached 0.1 AU, 10 microL aliquots were transferred to in 96-well plates containing 140 microL of BHIB broth supplemented with glucose (10 g/L) and a range of SPH03 covering from 0 to 3.2 g/L. The plate was incubated at 37° C. during 24 h. Then, the culture was removed of each well in the plate, followed by gentle washing with PBS buffer in order to remove free planktonic cells. The plate containing the bacterial biofilms was incubated 1 h at 60° C., followed by the addition of 200 microL of aqueous 4% crystal violet solution to each well, followed by incubation at room temperature during 15 min. The plate was washed and dried at 60° C. during 15 min. To dissolve the remaining crystal violet attached to biofilms, 200 microL of acetic acid 0.33% were added to each well, and the absorbance measured at 570 nm. The resultant absorbance measured in each condition is proportional to the amount of biofilm in the corresponding well. The results show a significant effect of SPH03 at concentrations higher than 1.9 g/L when compared with control cells (FIG. 21).

1. Method for preparing of an extract of 3-O-monodesmosidic saponins having formula (I) 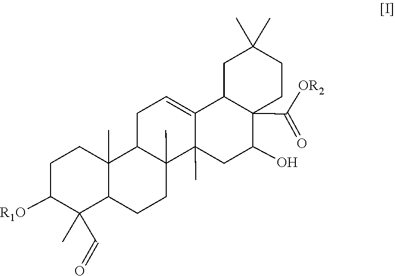
wherein R₁ is {xylopyranosyl-(1-+3)-glucopyranosiduronic acid} or a salt thereof, {xylopyranosyl-(1-+3)-glucopyranosiduronate salt}; {rhamnopyranosyl-(1-+3)-glucopyranosiduronic acid} or a salt thereof, {rhamnopyranosyl-(1-+3)-glucopyranosiduronate salt}; or {galactopyranosyl-(1-+2)-glucopyranosiduronic acid} or a salt thereof, {galactopyranosyl-(1-+2)-glucopyranosiduronate salt}; or {glucopyranosiduronic acid} or a salt thereof, {glucopyranosiduronate salt}, and R₂ is hydrogen or a cation specie, substantially free of bidesmosidic saponins, comprising:
a) obtaining a partially hydrolyzed extract from a plant biomass extract containing glycosides of quillaic acid and obtaining a crude hydrolysate,
b) recovering from the crude hydrolysate obtained in step a), an extract of monodesmosidic saponins comprising Compounds I and II, 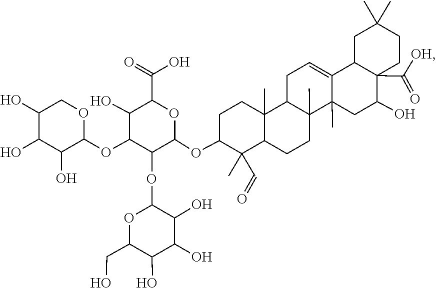
quillaic acid 3-O-{rhamnopyranosyl-(1-+3)-[galactopyranosyl-(1-+2)]-glucopyranosiduronic acid}, compound I, 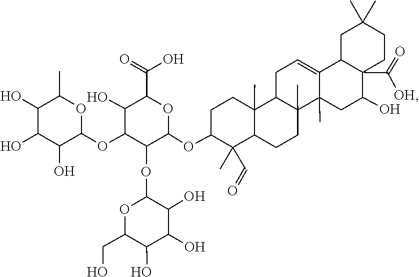
quillaic acid 3-O-{galactopyranosyl-(1-+2)-glucopyranosiduronic acid}, compound II;
c) culturing such extract obtained in step b) into a liquid fermentation culture of a microbial consortium (HNR2), wherein such microbial consortium (HNR2) comprising microorganisms selected from Rhodotorula sp having accession number RGM 3031 (colony P) dated Jul. 22, 2020 of the Chilean Collection of Microbial Genetic Resources (CChRGM); Microbacterium sp having accession number RGM 3021 (colony Y) dated Aug. 7, 2020 of the Chilean Collection of Microbial Genetic Resources (CChRGM); Mucor sp having accession number RGM 2984 (colony C) dated Aug. 7, 2020 of the Chilean Collection of Microbial Genetic Resources (CChRGM); Paenibacillus sp and having accession number RGM 3032 (colony W) dated Sep. 3, 2020 of the Chilean Collection of Microbial Genetic Resources (CChRGM), and
d) removing such microbial consortium (HNR2) from such fermentation broth obtained in step c) and recovering an Quillaja extract substantially free of microorganisms and bidesmosidic saponins.

2. The method of claim 1 wherein such plant biomass extract containing glycosides of quillaic acid is selected from an extract of bark, pruned branches and leaves of Quillaja plant.

3. The method of claim 2 wherein such Quillaja plant is selected from Quillaja saponaria Molina, Quillaja brasiliensis and Saponaria vaccaria.

4. The method of claim 2 wherein such extract of Quillaja plant is an extract of Quillaja saponaria Molina.

5. The method of claim 1 further comprising supplementing the fermentation broth with oligosaccharides of xylose at step c).

6. The method of claim 1 further comprising isolating such 3-O-monodesmosidic saponins having formula (I) from such extract substantially free of microorganisms and bidesmosidic saponins as resulting from step d), by solvent extraction followed by solvent evaporation at reduced pressure, or by chromatography followed by solvent removal at reduced pressure and lyophilization.

7. The method of claim 1 wherein such partially hydrolyzed extract of step a) is obtained from a plant biomass extract containing glycosides of quillaic acid selected of an aqueous Quillaja extract.

8. The method of claim 7 wherein such partially hydrolyzed extract of step a) is obtained by KOH hydrolysis from an aqueous Quillaja extract.

9. The method of claim 1 wherein such crude hydrolysate is obtained from submitting to aqueous ethanol, such partially hydrolyzed extract of Quillaja plant.

10. The method of claim 1 further comprising prior to step b), fractionating such crude hydrolysate to obtain a crude hydrolysate enriched in Compound I and Compound II.

11. The method of claim 10 wherein such fractionating is performed by chromatography on a hydrophobic resin.

12. An extract of 3-O-monodesmosidic saponins, substantially free of bidesmosidic saponins, stable at physiological pH and reduced hemolytic effect, having formula (I) 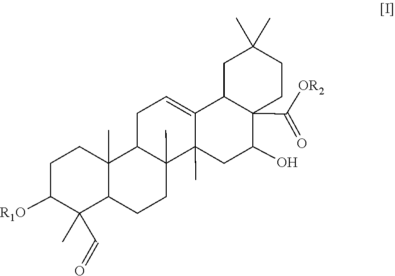
wherein R₁ is {xylopyranosyl-(1-+3)-glucopyranosiduronic acid} or a salt thereof, {xylopyranosyl-(1-+3)-glucopyranosiduronate salt}; {rhamnopyranosyl-(1-+3)-glucopyranosiduronic acid} or a salt thereof, {rhamnopyranosyl-(1-+3)-glucopyranosiduronate salt}; or {galactopyranosyl-(1-+2)-glucopyranosiduronic acid} or a salt thereof, {galactopyranosyl-(1-+2)-glucopyranosiduronate salt}; or {glucopyranosiduronic acid} or a salt thereof, {glucopyranosiduronate salt}, and R₂ is hydrogen or a cation specie.

13. A microbial consortium to culturing Quillaja extracts comprising microorganisms selected from Rhodotorula sp having accession number RGM 3031 (colony P) dated Jul. 22, 2020 of the Chilean Collection of Microbial Genetic Resources (CChRGM); Microbacterium sp having accession number RGM 3021 (colony Y) dated Aug. 7, 2020 of the Chilean Collection of Microbial Genetic Resources (CChRGM); Mucor sp having accession number RGM 2984 (colony C) dated Aug. 7, 2020 of the Chilean Collection of Microbial Genetic Resources (CChRGM); Paenibacillus sp and having accession number RGM 3032 (colony W) dated Sep. 3, 2020 of the Chilean Collection of Microbial Genetic Resources (CChRGM).

14. The microbial consortium of claim 13 further comprising one or more of Paenibacillus amylolyticus, Paenibacillus tundra, Paenibacillus tylopili, Microbacterium hydrocarbonoxydans, Microbacterium luteolum, Microbacterium phyllosphaerae, Rhodotorula mucilaginosa, Mucor rcemosus, Cylindrocarpon spp., Aspergillus niger and Aspergillus parasiticus.

15. A method for treating cancer comprising contacting cancer cells with an extract as defined in claim 12.

16. The method of claim 15 wherein such cancer cells are gastric cancer cells.

17. The method of claim 16 wherein such gastric cancer cells are adenoma cells and carcinoma cells.

18. A method for treating oral infections produced by oral pathogens comprising contacting cancer cells with an extract as defined in claim 12.

19. The method of claim 18 wherein such oral pathogens is Streptococcus mutants.
